(19) United States
(12) Patent Application Publication
(10) Pub. No.: US 2008/0300798 A1
(43) Pub. Date: Dec. 4, 2008

(54) CARDIBIOINDEX/CARDIBIOSCORE AND UTILITY OF SALIVARY PROTEOME IN CARDIOVASCULAR DIAGNOSTICS

(76) Inventors: John T. McDevitt, Austin, TX (US); Nicolaos Christodoulides, Austin, TX (US); Pierre N. Floriano, Austin, TX (US); Craig S. Miller, Lexington, KY (US); Jeffrey L. Ebersole, Lexington, KY (US)

Correspondence Address: FULBRIGHT & JAWORSKI L.L.P., 600 CONGRESS AVE., SUITE 2400, AUSTIN, TX, 78701, US

(21) Appl. No.: 12/104,303

(22) Filed: Apr. 16, 2008

Related U.S. Application Data
(60) Provisional application No. 60/912,123, filed on Apr. 16, 2007.

(51) Int. Cl. G01N 33/50

(52) U.S. Cl. 702/19

(57) ABSTRACT

Embodiments of the invention include methods by which cardiac biomarkers are assigned an index (cardiovascular biomarker index-cardiobioindex, CBI) as a means to describe the utility of each biomarker, or combination of biomarkers for risk evaluation, diagnosis or prognosis of cardiovascular disease status.

This application claims priority to U.S. Provisional Patent Application 60/912,123 filed on Apr. 16, 2007, which is hereby incorporated by reference in its entirety.

This invention was made with government support under grant number 9 R01 EB000549-04A1 and 5 U01DE015017 awarded by the National Institute of Health. Consequently, the government has certain rights in the invention.

### BACKGROUND OF THE INVENTION

I. Field of the Invention

The present invention relates generally to the fields of medicine, physiology, diagnostics, and biochemistry. In certain embodiments, the invention relates to assessment of biomarkers indicative of cardiovascular disease (CVD).

II. Background

Cardiovascular disease (CVD), having enormous health, social, and economical consequences, is the leading cause of death in developed countries. In the United States alone, the projected cost of CVD in 2005 is estimated at $431.8 billion, including health care services, medications, and lost productivity. Atherosclerotic Heart Disease (ASHD) or coronary artery disease (CAD), a cardiovascular disease condition, develops when lipids and inflammatory cells accumulate in the walls of coronary arteries, forming atherosclerotic plaques. As CAD progresses, clinical manifestations may develop, including the occurrence of angina. Acute Coronary Syndrome (ACS), which includes unstable angina and acute myocardial infarction (AMI), is associated with plaque rupture and thrombus formation in a coronary vessel, resulting in myocardial ischemia and often necrosis.

According to the American Heart Association (Heart and Disease Statistics-2004), the following dire morbidity and mortality statistics are associated with CAD in the United States: CAD is the primary cause of death in America today and was responsible for more than one third of U.S. deaths in 2004. Further, 13.2 million people (7.2 million males and 6.0 million females) living today have experienced a heart attack, angina or both, approximately 330,000 people a year will die of an ACS event inside or outside of the emergency room and 1.2 million Americans are expected to have a new or recurrent coronary event this year. In 2008, an estimated 770,000 Americans will have a new coronary attack, and about 430,000 will have a recurrent attack. It is estimated that an additional 175,000 silent first myocardial infarctions occur each year. Here, about every 26 seconds, an American will have a coronary event, and about every minute someone will die from a coronary event.

Despite enormous advances in genomics in the past decade that have produced a great number of microarray databases, data analysis procedures and protocols generated, cardiovascular disease proteomics is still in its infancy (Arab et al., 2006; Donahue et al., 2006; Huang, 2001; Jung et al., 2006; Lam et al., 2006; Mayr et al., 2006; Napoli et al., 2003; Stephan et al, 2006; Vasan, 2006; Verhoeckx et al., 2004; Curtis et al., 2005; Do and Choi, 2006; Fu and Van Eyk, 2006; Fung et al., 2005; Herrmann, 2003; Lee et al., 2007; Liszewski, 2006; Quackenbush, 2002; Zhu, et al., 2006). Clearly, there is a missing link between the areas of biomarker discovery and biomarker validation. In the past decade, hundreds of novel ASHD biomarkers have been identified, but the results of numerous research efforts have not yet changed clinical practice in a significant manner, simply because the vast majority of the discovered biomarkers have not yet been validated or selected. Another major limitation of the current biomarker validation approach is the lack of a common assay platform that allows for a multi-marker testing strategy. Unfortunately, the present scientific and medical communities are faced with disjointed information based on non-standardized data and multiple disparate test results achieved on separate instruments.

Understanding the complex pathobiology of CVD and applying that knowledge in assessing the risk and timing of future acute coronary events will help develop improved diagnostic tests and thus prevent or minimize some of the adverse outcomes of cardiac disease. There is a need for additional methods for biomarker identification and validation, as well as methods for diagnosing and prognosing various CVDs.

### SUMMARY OF THE INVENTION

Embodiments of the invention include methods by which factors, such as serum and saliva cardiac biomarkers, may be assigned an index (e.g., cardiovascular biomarker index-cardiobioindex/CBI) as a means to describe the utility of each biomarker, or combination of biomarkers, in a sample (e.g., a bodily fluid) to discriminate healthy individuals from cardiac disease patients. CBI may be derived from logistic regression analysis and may be defined by the area under the curve (AUC) from receiver operating characteristics (ROC) analysis. In certain aspects, biomarkers are validated and selected to achieve a particular efficacy or robustness in diagnosis and/or prognosis. In still further aspects, biomarker are assessed on a common platform. In yet a further aspect, biomarkers are assessed or evaluated concurrently. In certain aspects, biomarkers are assessed concurrently and on a platform comprising normalization and evaluation controls such as concentration titers of biomarker being measured. In further aspects, one or more biomarkers in a sample may be detected, measured or quantified by a detection device or system, e.g., lab-on-a-chip.

As used herein, biomarkers are substances used as indicators of a biologic state. It has a characteristic that is objectively measured and evaluated as an indicator of normal biologic processes, pathogenic processes, or pharmacologic responses to a therapeutic intervention. In certain aspects, biomarkers are proteins, protein fragments, or polypeptides. An index as it relates the present invention can indicate the relation of a value of a variable (or group of variables) to a base level. The base level is set so that the index produces numbers that are easy to understand and compare. Indices are used to report on a wide variety of variables.

These processes help identify important biomarkers relevant to (a) classification of risk for CAD, (b) AMI diagnosis and (c) AMI prognosis. Once biomarkers (BMs) with high CBIs are identified, a trained algorithm can be challenged with the measurements of selected biomarkers in healthy controls and cardiac disease patients. Here, threshold concentrations for yes or no tests (e.g., AMI diagnosis) or quartiles for RISK for 1st or recurrent event (low, medium low, high and very high) can be established. Once thresholds are established, tests may be applied for a general population using selected biomarkers to deliver a cardiobioscore (CBScore). In certain aspects, the CBScore is mathematically derived from the contributions of multiple biomarkers of risk/diagnosis and their CBIs to derive the cardiac health status of each subject tested. The CBIs can be used to define the method that included the selection of the biomarkers and the weighting factors that are associated with each of these biomarkers. This CBI definition process may occur after a clinical trial is completed and serve as a best fit to define the patient classification methodology. The CBI thus covers classification over a large patient group. An established CBI method can be used to score the individual patients cardiac health status. The latter method of providing diagnostic information to the individual patient is the CBScore.

All of this may be done in a non-invasive fashion at the point-of-care using saliva and lab on a chip (LOC) technology. Lab on a chip technology as well as point of care apparatus and sampling methodology can be found in various PCT publications, each of which are incorporated herein by reference in their entirety and include WO 2005/059551, WO 2007/002480, WO 2001/055702, WO 2007/005666, WO 2005/085855, WO 2003/090605, WO 2005/085854, WO 2005/090983, WO 2005/083423, WO 2000/004372, WO 2001/006253, WO 2001/006244, WO 2001/006239, WO 2001/055952, WO 2001/055701, WO 2001/055703, WO 2001/055704, WO 2002/061392, WO 2004/009840, WO 2004/072097, WO 2004/072613, WO 2005/085796, WO 2007/134191, and WO 2007/134189.

In certain embodiments, there may be provided methods for assessing cardiovascular disease status in a subject comprising the steps of: (a) measuring a biomarker level in a sample from a subject, wherein the biomarker is two or more of CRP, IL1β, IL-13, cTnI, BNP, FABP, CK-MB, IL-6, IL-8, IL-10, TNF-α, CD40L, IFN-γ, myoglobin, MMP9, sICAM-1, myeloperoxidase, IL-4, and/or IL-5; (b) evaluating biomarker levels with respect to a scoring index, wherein evaluation comprises: (i) assigning an index to each biomarker or combination of biomarkers based on its/their measured capacity to discriminate between cardiac healthy subjects and cardiac disease patients, and (ii) establishing a threshold level of biomarkers with the index greater than 0.5, 0.6, 0.7, 0.75, 0.8, 0.85, 0.90, 0.95, 0.98, 0.99, including all ranges and values there between, to discriminate cardiac healthy subjects from cardiac disease patients; and (c) determining a value representative of the cardiovascular disease status of the subject based on the evaluation of subject's biomarkers.

For example, assessment of cardiovascular status can include, but is not limited to, classification of risk for cardiovascular disease, diagnosis of acute myocardial infarction (AMI), assessment of risk for a second AMI, and/or patient prognosis after AMI. In certain aspects, AMI diagnosis in serum includes evaluation of cTnI, CK-MB, BNP, myoglobin, CRP, including all or combinations of 2, 3, or 4 of these biomarkers may be used; for AMI diagnosis in saliva, evaluation of CRP, IL-1β, myeloperoxidase, myoglobin, MMP9, sICAM-1, or combination of 2, 3, 4, 5, or 6 of these biomarkers can be used. In certain aspects the sample is a serum sample, a saliva sample, and/or a stimulated saliva sample.

In a further embodiment, the threshold level for a biomarker may indicate the presence or absence of a biomarker, or indicate a risk level division in which the measured biomarker level falls. In certain aspects, the threshold level can be determined by the steps of: (a) obtaining a sample from each of a plurality of subjects including cardiac healthy subjects and cardiac disease subjects at risk of or having cardiovascular disease; (b) quantifying the level of the biomarkers in each sample; (c) comparing the level between the cardiac healthy subjects and the cardiac disease subjects; (d) identifying and selecting a biomarker that distinguish the cardiac healthy subjects from the cardiac disease subjects; and (e) determining a threshold level for the selected biomarker based on discriminatory concentration for the selected biomarker (e.g., that level that distinguishes between the two groups at a particular relevance).

In still a further embodiment, there are provided methods of establishing a cardiobioindex comprising the steps of: (a) obtaining a plurality of samples from a first and second population of subjects, wherein the first population has a normal cardiac status and the second population has a cardiovascular condition; (b) quantifying the level of a factor in each sample, optionally by a detection device, such as a lab-on-a-chip (LOC); (c) comparing the levels of the factor between the healthy subjects and the cardiac patients; and (d) determining the cardiobioindex of the factor by logistic regression and ROC analyses; and (e) utilizing factors or biomarkers with in the cardiobioindex greater than 0.6, 0.65, 0.7, 0.75, 0.8, 0.85, 0.9, 0.95, 0.98, or 0.99, including all values and ranges there between, for cardiac diagnostics.

For example, the factor may be BMI (body mass index), blood pressure, total cholesterol, lipid ratio or a combination thereof, or a biomarker.

Biomarkers include, but are not limited to, LDL, HDL, C-reactive protein (CRP), adiponectin, Apolipoprotein A (ApoA), Apolipoprotein B (Apo B), E-selectin, IL-1α, IL-1β, IL-4, IL-5, IL-6, IL-1β, IL-10, IL-13, IL-18, creatinine kinase-MB (CK-MB), B-natriuretic peptide (BNP), FABP (cardiac fatty acid protein), TNF-α, MCP-1, MMP-9, MPO, Intercellular Adhesion Molecule (ICAM), Vascular Cellular Adhesion Molecule (VCAM), sCD40L, ENA78, fractalkline, PIGF, PAPP-A, RANTES, sCD40L, vWF, D-dimer, IMA, FFAu, Choline, cTnT, Cardiac troponin I (cTnI), Myoglobin, NT-proBNP, MMP or a combination thereof.

In certain aspects, the cardiovascular disease (CVD) could be atherosclerotic heart disease, acute coronary syndrome, cardiomyopathy, microvascular angina, hypertension, ST elevated myocardial infarction, non-ST elevated myocardial infarction, acute myocardial infarction (AMI), coronary heart disease (CHD) or coronary artery disease (CAD). In further aspects, the sample may be a body fluid, such as serum, saliva, urine, blood, blood plasma, or cerebrospinal fluid.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Illustrates a cardiac cascade with specific protein biomarkers at various stages of disease.

FIG. 2. Illustrates a multi-marker screening approach that provides improved risk stratification in CAD. Each biomarker (C-reactive protein, troponin I and B-natriuretic protein) provides insight into a different pathophysiological mechanism. Simultaneous assessment of all three biomarkers yields complimentary prognostic information.

FIG. 3. Log NT-proBNP values across CAC score categories. P<0.0001.

FIG. 4. Individual and joint risks (hazard ratios-HR) for recurrent coronary events for patients in high- and low-risks partitions for D-dimer, ApoA-I and ApoB.

FIG. 5. Diagnosis of AMD Background

FIG. 6. Cardiac array images of extreme phenotypes (healthy and cardiac) using selected cardiac biomarkers (IL-1β, IL-13, cTnI, BNP, FABP, CKMB, IL-6, IL-8, IL-10, TNF-α, CD40L, IFN-γ, IL-4 and IL-5).

FIG. 7. An example of preliminary evaluation of an established serum cardiac risk factor (poor separation— e.g., total cholesterol-ACA study). (i) Healthy and At risk—Cut off: 10; Sensitivity=20/23—86.96%/Specificity=1/7—14.29%/Accuracy=21/30—70%; (ii) Healthy and Cardiac Patients—Cut off: 10; Sensitivity=17/20—85%/Specificity=1/7—14.29%/Accuracy=18/27—67.67%.

FIG. 8. Another example of preliminary evaluation of an established serum cardiac risk factor (excellent separation, e.g., Serum hsCRP). Healthy (H) and Cardiac Patients (U)—Cut off: 25; Sensitivity=13/13—100%/Specificity=22/22—100%/Accuracy=35/35—100%.

FIG. 9. Preliminary evaluation of single salivary biomarkers: an example of a salivary BM with low discrimination capabilities. Healthy (H) and Cardiac Patients (U)—Cut off: 0.6; CBI: Sensitivity=8/11—73%/Specificity=4/10—40%/Accuracy=12/21—57%.

FIG. 10. Preliminary evaluation of single salivary biomarkers: an example of a salivary BM with high cardibioscore. Healthy (H) and Cardiac Patients (U)—Cut off: 20; Sensitivity=8/11—73%/Specificity=8/10—80%/Accuracy=16/21—77%.

FIG. 11. Preliminary evaluation of aggregated salivary biomarkers. Healthy (H) and Cardiac Patients (U)—Cut off: 40; Sensitivity=10/11—91%/Specificity=6/10—60%/Accuracy=16/21—77%.

FIG. 12. Preliminary evaluation of Aggregated for selected salivary biomarkers. Healthy (H) and Cardiac Patients (U)—Cut off: 5; Sensitivity=10/11—91%/Specificity=5/10—60%/Accuracy=15/21—72%.

FIG. 13. Comparison of specificity/sensitivity/accuracy of different combinations of salivary biomarkers.

FIGS. 14A-14C. The three stages involved in the development of a new diagnostic test. The first step involves the discovery of the new biomarkers. Modern advances in proteomics discovery tools have led to the development of several proteomics methods that have played a central role in the identification of disease biomarkers associated with CVD. Here mass spectrometry has become a central tool that is used in connection with a wide variety of separation methods such as 2-D gel electrophoresis, liquid chromatography, ion exchange and reverse phase chromatography, (FIG. 14A). Following the discovery it is necessary to exploit a second set of tools that can be used to explore these patient-to-patient differences en route to defining the efficacy of the new biomarkers. Here, clinical trials that focus on the disease progression as a function of biomarker expression levels are required to validate these biomarkers (FIG. 14B). Critical for the validation step is the use of high throughput methodologies (ELISA and LOC) that can be used to explore the expression levels across the diseased and healthy populations. Shown in IC are examples of assay platforms that may be suitable for this final step. Here, lateral flow immunoassay kits have been popular for cases where a more limited number of biomarkers are sufficient. On the other hand, the bead-based lab-on-a-chip systems (bottom panel of FIG. 14C) may serve as a better fit for future clinical testing where multiple cardiac biomarkers are measured concurrently. Here, a series of cardiac-specific μ-chips for the multiplexed testing of biomarker panels for CVD have been developed (Christodoulides et al., 2002; Christodoulides et al., 2005b; Christodoulides et al., 2005a). The use of the bead-based LOC approach in both the validation and clinical phases is expected to increase the efficiency of the translation of the new assays into clinical practice.

FIG. 15. Wilcoxon plot demonstrating the relative concentration range of a number of salivary biomarkers for control and disease, with respect to cardiac disease, patient groups, as measured by μ-array and LOC methods. Here color boxes describe data comprised between the 25-75th percentile, Whisker boxes describe data between the 10-90th percentile, line in color box describes the median, filled circles are the outliers.

FIGS. 16A-16D. The mechanics for the development of the cardiobioindex: FIG. 16A: Measure biomarker and record data. FIG. 16B: Use a dichotomous approach to divide the sample population into a “control” and “diseased” population, the latter encompassing various sub-categories of cardiovascular disease; FIG. 16C: Use logistic regression to assess the importance/relevance of biomarkers to cardiovascular disease. Derive cardiobioindex by using the area under the ROC curve, or the C-statistic. FIG. 16D: With this ranked evaluation for both the diseased and control populations (line indicates mean values of biomarker for the two groups studied), it is possible to select threshold values from which the sensitivity and specificity for this particular biomarker index may be derived.

FIGS. 17A-17D. Validation of cardiobioindex method with established serum risk factors of cardiac disease. (FIG. 17A) Serum classifiers of cardiac disease with varying input cardiobioindex values; here, each spoke in the graph represents measure of the cardiobioindex for biomarker indicated. For example the cardiobioindices for HDL and CRP were measured at 0.8, while the cardiobioindex for LDL was calculated as 0.671. (FIG. 17B) ROC curves for CRP and TC/HDL, (FIG. 17C) classification of control and cardiac disease patients by TC/HDL and (FIG. 17D) classification of control and cardiac disease patients by CRP. Line indicates mean values of biomarker for the two groups studied; second line indicates threshold value from which values for sensitivity and specificity are derived.

FIG. 18. The cardiobioindex for a set of individual CVD biomarkers, as measured by Luminex® (IL-1β, IL-6, MCP-1, RANTES, TNF-α, CRP, adiponectin, E-selectin, MMP-9, MPO, sICAM-1, sVCAM-1, fractalkine, and sCD-40), ELISA (ENA-78 and IL-18) and LOC*(CRP), within the context of saliva measurements.

FIG. 19. The biomarker CRP achieves a superior cardiobioindex when measured with the more sensitive LOC method than with Luminex®. The Luminex® approach provides a cardiobioindex for CRP of 0.661 ((SE 0.1888, p-value 0.1973 and 95% confidence interval 0.291-1.000), while the counterpart LOC method achieved a cardiobioindex of 0.929 (SE 0.0821, p-value <0.0001 and 95% confidence interval 0.768-1.000)

FIGS. 20A-20C. Cardiobioindex for single and aggregate salivary biomarkers of cardiac disease. (FIG. 20A) Here, single biomarkers IL-1β, IL-13, BNP, IL-6, TNF-α, IL-10, IL-4, sCD40L, IL-8 and IL-5 (as measured by proteomic μ-array chip) and CRP (as measured by LOC) produced cardiobioindices in the range of 0.534-0.665, while their combination, as reflected by the biomarker panel (BM panel), resulted in a significantly improved cardiobioindex of 0.932 (SE 0.0574, p-value <0.001 and 95% confidence interval 0.819-1.000). (FIG. 20B) Here, the combination of all of the fore-mentioned biomarkers contributes to the identification of a superior cardiobioindex and allows for the classification of control and cardiac disease patients with 91% sensitivity and 80% specificity. (FIG. 20C) Multiplexed detection of cardiac biomarkers in saliva by LOC method.

FIG. 21. Wilcoxon box and whisker plot demonstrating the relative concentration range of salivary biomarkers for control and diseased, with respect to ASHD, patient groups, as measured by μ-array proteomic chip and LOC methods. Here, color boxes describe data between the 25-75th percentiles, Whisker boxes describe data between the 10-90th percentiles, line in color box describes the median value, and filled circles are the outliers.

FIG. 22. Performance of single and aggregate salivary biomarkers for the classification of ASHD. Single biomarkers TNF-α, sCD40L, BNP, IL-1β, IL-4, IL-5, IL-6, IL-8, IL-10, IL-13 (as measured by proteomic μ-chip) and CRP (as measured by LOC) as measured in unstimulated saliva produced cardiobioindices in the range of 0534-0.665, while their combination, i.e., BM panel, resulted in a significantly improved cardiobioindex of 0.932 (SE 0.0574, p-value <0.001 and 95% CI 0.819-1.000). Here, the combination of TNF-α, sCD40L, BNP, IL-1β, IL-4, IL-5, IL-6, IL-8, IL-10, IL-13 and CRP biomarkers contributes to a superior classification of healthy controls and CAD patients with 91% sensitivity and 88% specificity.

FIG. 23. The biomarker CRP as measured in stimulated saliva achieves superior classification of healthy controls and ASHD patients when measured with the more sensitive LOC system than with Luminex®(g.

FIG. 24. Multi-analyte testing capacity of LOC system. Here, 8 cardiac biomarkers (CRP, sCD40L, HSA, IL-1β, IL-6, MCP-1, MPO and TNF-α) are detected concurrently, in one assay run, by their corresponding LOC bead sensors arrayed in triple redundancy. Signals derived on negative control beads (neg) and LOC calibrator beads (cal) are also shown.

FIG. 25. Comparison of the relative levels of 21 proteins as measured in the serum and unstimulated saliva (UWS) samples.

FIG. 26. Mean analyte levels of 9 biomarkers in serum of AMI and healthy controls.

FIG. 27. Mean analyte levels of 9 biomarkers in unstimulated saliva (UWS) of AMI and healthy controls.

FIG. 28. Ratio of median concentration for the ACS(NSTEMI and STEMI) over median concentration for the controls.

FIG. 29. CBI (cardiobioindex) of some top ranking biomarkers in saliva by logistics regression and ROC analysis of representative data.

FIG. 30. Multiplexed test of the LOC sensor.

FIG. 31. Saliva-based test of top ranking biomarkers (CRP and MPO) in conjunction with EKG in saliva compared with serum-based tests.

FIGS. 32A-32B. Diagnostics of AMI and ACS using Myoglobin threshold value. FIG. 32A. Diagnostics of AMI subjects (STEMI and NSTEMI). FIG. 32B. Diagnostics of ACS.

FIG. 33. CBI of myoglobin paired with CRP in the UWS.

### DETAILED DESCRIPTION

Having realized that the current approaches of evaluating cardiac biomarkers are, for the most part, qualitative and, thus, limiting, the inventors developed this method by which cardiac biomarkers, for example, serum and saliva cardiac biomarkers, are assigned an index (cardiobioindex), which may be used to describe the ability of the biomarker (or combination of biomarkers) to discriminate between healthy individuals and cardiac disease patients. Here, the relative attributes of the individual biomarkers can be assessed as well as the utility of the various combinations. Further, the scores are normalized so that the biomarker concentration range can be accounted for.

Furthermore, even though this method can be applied for serum, which has been the traditional diagnostic fluid for cardiac diagnostics, additional aspects of the disclosure relies in the utility of saliva as a diagnostic fluid for cardiovascular disease. Other biologic fluids and samples are contemplated.

The inventors describe a method for the classification and diagnosis of cardiovascular disease utilizing body fluids, such as salivary and blood fluids, and using proteins found within these fluids as cardiac biomarkers. The method assigns a numerical score, defined here as a CARDIac BIOmarker INDEX (i.e., “cardiobioindex”), to each, and/or a combination, of biomarkers, as measured by a variety of detection/measurement methods. The cardiobioindex (CBI) is a reflection of the sensitivity, specificity, and overall accuracy of the salivary/blood biomarker(s), derived from logistic regression and defined by the area under the curve (AUC) from receiver operating characteristics (ROC) analysis. CBI describes the capacity of a biomarker (or combination of biomarkers) to classify healthy and cardiac patients. It is intended to promote cardiac biomarker-based diagnostics in saliva and saliva with respect to the following 3 areas relevant to cardiac diagnostics: (A) Classification of coronary artery disease (CAD), (B) Diagnosis of acute myocardial infarction (AMI), and/or (C) Prognosis of AMI.

As used herein, diagnosis or diagnostics is the process of identifying a medical condition or disease by its signs, symptoms, and from the results of various diagnostic procedures. The conclusion reached through this process is called a diagnosis. The term “diagnostic criteria” (e.g., cardiobioscore related to a cardiobioindex) designates the combination of signs, symptoms, and test results that allows one, e.g., a physician, to ascertain the diagnosis of the respective disease. Prognosis is a term denoting a prediction of how a patient's disease will progress, and whether there is chance of recovery. Prognosis includes methods of predicting how a patient (given their condition) may respond to treatment. Symptoms and tests may indicate favorable treatment with standard therapies. Likewise, a number of symptoms, health factors, and tests may indicate a less favorable treatment result with standard treatment (treatment prognosis)—this may indicate that a more aggressive treatment plan may be desired.

This method is a non-invasive, pain-free assessment/classification of cardiac risk using saliva as a diagnostic fluid, which, when used in conjunction with a point of care device, introduces the possibility of a home-based cardiac assessment test.

This method includes, but is not limited to methods for: (i) Validation of existing (established), emerging and novel cardiac biomarkers; (ii) Application of sensitive and quantitative assays for the detection/measurement of cardiac biomarkers in saliva; (iii) Definition of a fingerprint of cardiac disease through a saliva/serum-based multi-marker screening strategy; (iv) Introduction of a point-of-care device that will host/integrate above features for the assessment of cardiac risk both in whole blood, plasma, serum and saliva.

The methods described can be completed at the point-of-care enabling more rapid and effective diagnosis of cardiovascular disease and reduction of health care costs, while at the same time, improving the diagnostic utility of cardiac biomarkers is one aspect of the methods.

The use of the Cardiobioindex (CBI) for protein (proteomic) biomarkers found in both serum and saliva for diagnostic and prognostic applications is described herein. The Cardiobioindex could also be used to gauge the efficacy of treatment and guide future therapy. Further uses of the method are contemplated that target cellular and/or genomic targets/biomarkers, in serum, saliva and other bodily fluids, such as urine and cerebrospinal fluid. Additionally, the same or similar biomarker scoring method may be applied for diagnostics/classification of patients of other disease states, such as cancer, autoimmune disease, etc.

### I. Utility of Lab-on-a-Chip (LOC) for Cardiac Classification and Risk Assessment at the Point of Care (POC)

Over the past five decades, the microelectronics industry has sustained tremendous growth and has become what is arguably the most dominant industrial sector for our society. The electronics industry has spawned annual growth of over 30% over this extended time period and has touched almost every aspect of our modern lives through the development of personal computers, portable communication devices, various consumer electronics, navigation tools, and imaging devices. The availability of a powerful microfabrication tool set that can be used to process these devices in a highly parallel manner has led to this explosive growth. Recently, it has become clear that the electronics industry will face new and significant challenges as component device feature sizes shrink into the nanometer size range. However, with the challenge here has come the opportunity to develop a number of fascinating new sensors and devices using nanometer sized building blocks. Challenges with spiraling health care costs, the global HIV crisis, environmental and homeland defense areas all provide strong motivation for the creation of a bridge between microelectronics, nano science-engineering and the health sciences. The ultimate applications to be derived from such interdisciplinary efforts are likely to occur for the sectors of life sciences and healthcare industries.

Indeed, remarkable advances have been made recently in the development of miniaturized sensing and analytical components for use in a variety of biomedical and clinical applications (Liu et al., 2003; Manz et al., 1990; Situma et al., 2005; Tudos et al., 2001; Verpoorte and De Rooij, 2003; Whitesides, 2005). However, the ability to assemble and interface individual components in order to achieve a high level of functionality in complete working devices continues to pose a daunting challenge for the scientific community as a whole. Lessons learned from the microelectronics and computer-software industries provide inspiration for what may be gained from the marriage of microelectronics and in vitro diagnostics areas. Indeed, there are some interesting parallels between the current state of medical devices, in particular, in vitro diagnostics, and the evolution of microelectronics. While medical tests have traditionally been completed in central laboratories that are filled with specialized equipment and trained technicians, there is currently a trend to complete more and more tests using portable instrumentation. Indeed, the point-of-care medical device area represents now the fastest growing sector of in vitro diagnostics.

Tremendous advances have been made recently in the area of LOC devices exploiting the advantages of miniaturization mediated by the small reagent and sample volumes required. Smaller sample and reagent volumes translate to rapid analysis times and less waste volumes, and result in more cost-effective assays that can be operated with less technological constraints making them suitable as a high throughput biomarker validation tool and amenable to point-of-care testing (POCT) (Tudos et al., 2001). Most importantly, these characteristics, when fully developed into a functional system, have the potential to lead to a significant reduction in the time that is needed for an accurate biomarker testing for the diagnosis and subsequent treatment of heart disease.

The inventors have combined and adapted the tools of the nano materials and microelectronics for the practical implementation of miniaturized sensors that are suitable for a variety of important applications. The performance metrics of these miniaturized sensor systems have been shown to correlate closely with established macroscopic gold standard methods, making them suitable for use as subcomponents of highly functional detection systems for analysis of complex fluid samples. These efforts remain unique in terms of functional LOC methods having a demonstrated capacity to meet or exceed the analytical characteristics (sensitivity, selectivity, assay variance, limit of detection) of mature macroscopic instrumentation for a variety of analyte systems including: pH, DNA oligonucleotides, metal cations, biological co-factors, and inflammation markers in serum and saliva (Christodoulides et al., 2002; Curey et al., 2001; Goodey et al., 2001; Goodey and McDevitt, 2003; Lavigne et al., 1998; McCleskey et al., 2003a; McCleskey et al., 2003b; Wiskur et al., 2003; Ali et al, 2003; Rodriguez et al., 2005; Christodoulides et al., 2005a; Floriano et al., 2005; Li et al., 2005a; Christodoulides et al, 2005b; Li et al., 2005b).

Having demonstrated the functionality of the subcomponent systems for miniaturized sensor systems, it becomes important now to search for effective strategies that would enable the translation of such promising miniaturized sensor concepts into important clinical applications. Only with the early implementation of the mini-assay systems for real-world clinical testing will the modular assay system be developed in a manner that will service the future needs of clinicians and the research communities. While the ultimate goal of such research endeavors is to develop universal assay systems that can be reprogrammed rapidly for new application, the steps taken here will target the development of a flexible biomarker validation tool that can support clinical research and clinical treatment of patients with heart disease, the number one health problem in developed countries.

As a clinical research tool, the LOC device offers the ability to perform multiplex assays in small sample volumes. Additionally, the versatility of this system and its demonstrated enhanced sensitivity makes it more a more sensitive biomarker validation tool, while at the same time amenable to applications involving a variety of bodily fluids, such as saliva, in which the analyte concentration may be extremely low (Goodey et al., 2001; Christodoulides et al., 2005b). For example, salivary biomarkers that were previously undetectable by standard methods, may now be targeted with the UT LOC device to assess systemic disease in a non-invasive fashion (Christodoulides et al., 2005b).

Certain aspects of the present invention address the need for multiplexed, multi-class LOC assays for a more efficient screening, classification and staging of cardiac risk in both serum and saliva.

### II. Cardiac Biomarkers

In its initial, but crucial stages, CAD is indeed a silent disease whereby a series of molecular- and cellular-level events occur within the vasculature, long before the obvious clinical manifestations begin to appear. Unfortunately, the occurrence of ACS is most often unpredictable because the underlying events responsible for it frequently occur without any obvious clinical symptoms. In fact, not even coronary angiography, the current gold standard for diagnosis of CAD, is capable of identifying these events as this method only provides a negative image of the internal lumen of a blood vessel and lacks the capability to adequately evaluate the vessel wall where an atherosclerotic plaque actually develops (Nakamura et al., 2004).

Early medical intervention in high-risk individuals is an ideal way to combat ASHD. However, in current medical practice, CAD risk assessment tools fail to detect an alarmingly large number of such individuals that suffer significant pain, lose cardiac function and in some cases die. In many such cases, the adverse outcome can be prevented by early intervention with existing medication. Ultimately, since most of these risk factors are modifiable, their early identification is crucial to the survival of the patient. If a cardiac risk pattern (profile) is identified in a prompt, accurate and efficient way, then a highly specific secondary prevention drug regimen for cardiovascular disease can be applied (aspirin, statins, and beta-blockers and ACE-inhibitor therapies). Such treatments are modifiable on an individual basis as a means to prevent and thus alter the adverse outcome of a first cardiac event.

Although atherosclerosis was formally considered a bland lipid storage disease, major advances in basic, experimental and clinical science over the last decade established its strong association with inflammation. Insights gained from the link between inflammation and atherosclerosis have defined specific protein biomarkers, as well as cells, as independent risk factors for heart disease that can now yield predictive and prognostic information of considerable clinical utility (Libby et al., 2002).

In the last decade, there has been an explosion of scientific (basic and clinical) research that has contributed to an increased understanding of the specific mechanisms and pathological pathways that result in heart attacks. Inflammation has been identified as a major contributor to the heart disease process. Further, there have been a large number of important studies that have identified a plethora of relevant biomarkers with potential diagnostic and prognostic utility.

One such biomarker whose measurement in serum is now contributing in a significant manner to the understanding and diagnosis of CAD is C-reactive protein (CRP) (Libby et al., 2002; Ridker, 2004). The biomarker CRP was originally identified as a substance observed in the plasma of patients with acute infections that reacted with the pneumococcal C-polysaccharide. It is now classified as a characteristic acute phase reactant in human serum and a classic marker of inflammation (Kushner and Rzewnicki, 1994). This important inflammation marker is derived from the liver and interestingly, according to recent studies, from vascular endothelial cells (Venugopal et al., 2005). CRP production is regulated by cytokines, such as TNFα, IL-1β and IL-6. The biomarker IL-6, as the major initiator of the acute phase response, induces the synthesis of CRP, as well as that of other acute phase reactants (Baumann and Gauldie, 1990; Baumann et al., 1990; Depraetere et al., 1991; Ganaphthi et al., 1991; Ganter et al., 1989; Toniatti et al., 1990). Given the role of IL-6 in CRP regulation, the combined use of IL-6 and CRP protein levels as indicators of inflammation may provide a better prediction of risk associated with inflammation than would use of either indicator alone (Harris et al., 1999).

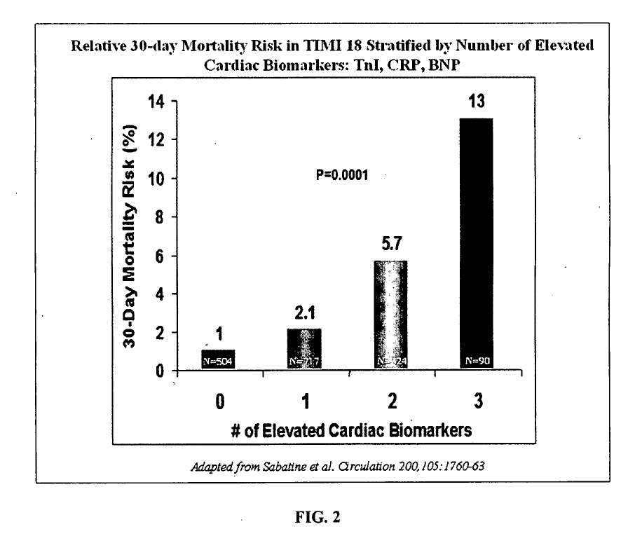

Interestingly, when biomarkers TnI, BNP, and CRP are used together, they enhance risk stratification compared with the use of these markers individually (Sabatine et al., 2002). These important studies demonstrate that a simple integer score in which 3 distinct biomarkers are evaluated provide excellent risk stratification in CAD (FIG. 2).

Cardiac biomarkers hold great promise as tools to better understand individual differences in the pathobiology of coronary artery disease (CAD), and may ultimately help individualize treatment strategies (Ridker et al., 2005). For example, in patients with ACS, creatinine kinase-MB and troponins have been firmly established as cardiac biomarkers of myocardial necrosis, which not only assist in the diagnosis of myocardial infarction (MI), but also help to direct treatment (Morrow et al., 2001). BNP serves as a marker of hemodynamic stress and neurohormonal activation in patients with acute and chronic CAD. The same biomarker is strongly associated with the development of death and heart failure, independent of clinical variables and levels of other biomarkers (de Lemos et al., 2001; Kragelund et al., 2005).

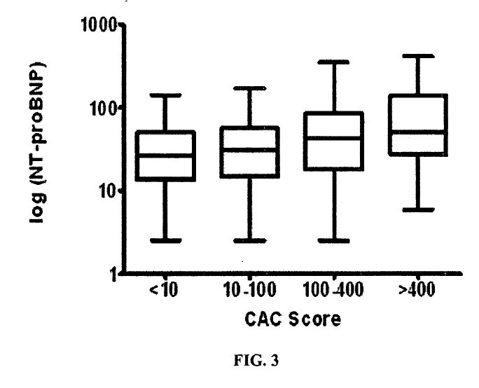

In heart failure, BNP and NT-proBNP, have been widely adopted as tools to facilitate heart failure diagnosis and risk stratification (de Lemos et al., 2003; Maisel et al., 2002). Indeed, BNP and NT-proBNP provide more powerful prediction of future risk than any other clinical or biomarker variables identified to date, with risk ratios for death of 3-4 associated with BNP elevation. BNP may help guide medical therapy based on outpatient monitoring. In addition, measurement of NT-proBNP in the Dallas Heart Study (DHS) showed that higher coronary artery calcium scores were independently associated with higher log NT-proBNP levels (p=0.03) (FIG. 3).

Recently, the potential additional value of troponins has been explored in patients with heart failure. As many as 50% of patients with decompensated heart failure will have evidence of troponin elevation at the time of presentation, and persistent elevation is identified in ˜20-25%. Troponin elevation is associated with excess risk for mortality, and provides incremental and additive prognostic information to BNP (Horwich et al., 2003). However, no single marker or combination of markers exists to adequately predict which patients will develop clinically significant HF or will progress to class IV HF with possible need for mechanical support or cardiac transplantation.

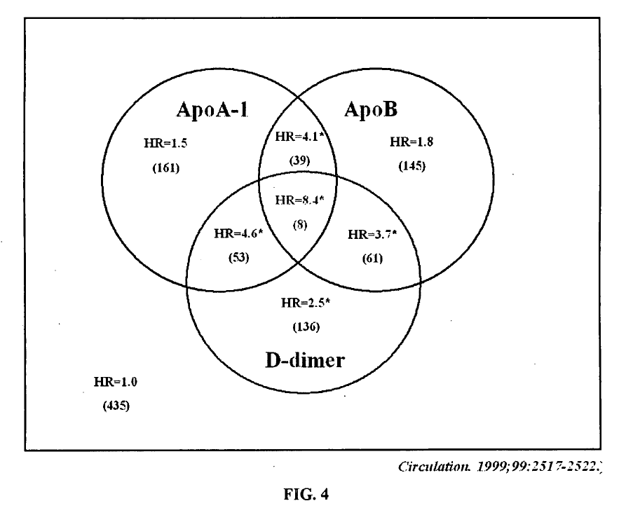

The presence of factors that reflect enhanced thrombogenic activity have also been shown to be associated with an increased risk of recurrent coronary events during long term follow up of patients who have recovered from myocardial infarction. Here, high levels of D-dimer (hazard ratio 2.43; 95% CI, 1.49 to 3.97) and apoB (hazard ratio 1.82; 95% CI, 1.10 to 3.00) and low levels of apoA-I (hazard ratio 1.84; 95%, 1.10 to 3.08) were independently associated with recurrent coronary events, indicating that a procoagulate and a disordered lipid transport contribute independently to recurrent coronary events in post-infarction patients. Most importantly, the risk associated with the combination of all 3 risk factors was multiplicative (FIG. 4).

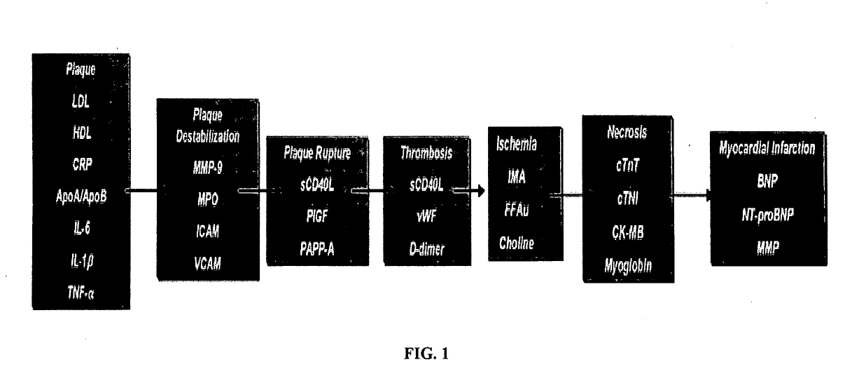

Several factors have converged to enhance interest in biomarkers in contemporary diagnostic cardiovascular medicine. First, considerable advances have been made in the understanding of the patho-physiological processes that contribute to various stages of cardiovascular disease. For example, as shown in FIG. 1, a significant number of protein biomarkers are identified as contributors to various stages of the cardiac cascade, from plaque formation to myocardial infarction (Vasan, 2006). Second, clinicians face an ever-increasing array of treatment options for patients with cardiovascular disease, and risk becoming overwhelmed by the number of choices they must make for common disorders. Many clinicians have become frustrated by the “one size fits all” approach advocated by guideline committees and staunch proponents of evidenced-based medicine. By providing a window into underlying patho-physiology, biomarkers offer the potential for guiding a more individualized approach to treatment of cardiovascular disease in the future. Finally, novel technologies now permit rapid identification and purification of high-affinity monoclonal antibodies against potentially important plasma proteins. High-throughput robotic assay methods have also been developed that allow performance of large-scale screening of stored blood samples in a relatively short period of time. Thus, both clinical demand for newer risk stratification tools and “supply” of novel biomarkers have increased concurrently. From this context, it is important to consider that blood-based tools for diagnosis and risk stratification in coronary disease are evolving in three parallel, and closely-associated, directions aimed for the analysis of circulating protein biomarkers, cell-surface markers and genetic polymorphisms.

Clearly, there is a missing link between the areas of biomarker discovery and biomarker validation. In the past decade, hundreds of novel ASHD biomarkers have been identified, but the results of numerous research efforts have not yet changed clinical practice in a significant manner, simply because the vast majority of the discovered biomarkers have not yet been validated (Anderson, 2005; Anderson and Anderson, 2002; Ludwig and Weinstein, 2005; Omenn, 2006;. Zolg, 2006). Another major limitation of the current biomarker validation approach is the lack of a common assay platform that allows for a multi-marker testing strategy that scans all three analyte classes. Unfortunately, the present scientific and medical communities are faced with disjointed information based on non-standardized data and multiple disparate test results achieved on separate instruments.

### III. Diagnosis of Acute Myocardial Infarction (AMI) Background

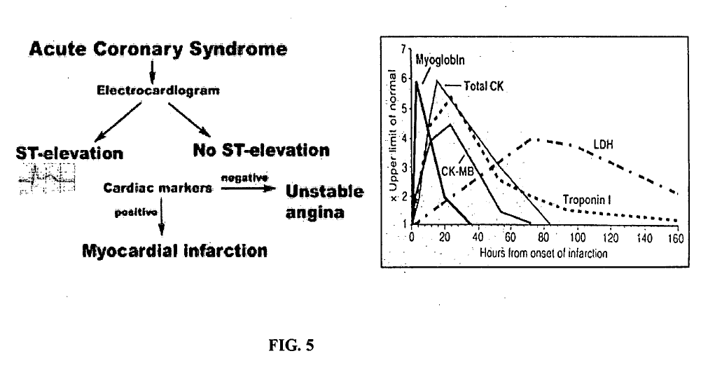

Currently, the diagnosis of AMI is usually predicated on the World Health Organization (WHO) criteria of chest pain, electrocardiogram (EKG) changes, and increases in blood levels of markers of myocardial injury (FIG. 5). Unfortunately, a significant number of AMI cases are missed or diagnosed late, while about half of the patients with “typical” symptoms do not have AMI.

The diagnosis of AMI is particularly difficult in the elderly, where relatively minor symptoms may reflect acute ischemia. The EKG is specific for AMI, but lacks sensitivity as it misses AMI cases with no ST-elevation, i.e. NSTEMI patients. The EKG also provides additional information regarding localization and the extent of the injury. However, sometimes, it is not easy to distinguish remote injury from a more recent one. In contrast, biochemical markers have excellent sensitivity for diagnosing AMI. By combining the most sensitive and the most specific tests, diagnostic accuracy can be enhanced.

The crucial step in ruling in/out the diagnosis of AMI is the measurement of myocardial enzymes in the serum. The rate of release of specific proteins differs depending on their intracellular location, molecular weight, and the local blood and lymphatic flow. The temporal pattern of marker protein release is obviously of diagnostic importance. Here, delays in patient entry from the onset of infarction may miss elevations of cardiac enzymes that are elevated early from the onset of infarction (e.g., myoglobin) which may affect the diagnosis and translate in delay of treatment (i.e., reperfusion), which ultimately could lead to increased mortality in myocardial infarction.

According to a recent report, emergency rooms are so overwhelmed with patients that it takes nearly an hour for 25% of heart attack victims to be seen by a doctor. During the 1997-to-2004 study period, as the number of emergency room visits rose and the number of emergency departments declined, the time it took for any patient to see a doctor stretched to 36% of the patients. But the increase was, in fact longer, to 40%, for patients identified by a triage nurse as needing help immediately. Surprisingly, the patients who saw the greatest increase in waiting time were ones whose lives most depend upon rapid treatment: those having a heart attack. Every minute of delay in treatment during a heart attack increases the likelihood that the patient will die, but heart attack patients waited 150% longer for care by the end of the study period, or 20 minutes on average. One in four waited 50 minutes or more. Added to that is the time the patient, or the close relative, took to call the emergency in, and the time it took to transport him/her to the ER. There is a need for improvement on minimizing the time delay between arrival at the emergency department and performance of reperfusion, by either pharmacological or catheter-based approaches.

Methods that make assessment easier, faster and predictable could indeed save lives. The new saliva-based microchip tests presented in certain embodiments of the instant invention promise new testing options that may help diagnose AMI in an earlier and more prompt fashion. The use of saliva in conjunction with the LOC (lab-on-a-chip) sensor promise to improve cardiac care.

### IV. Generation of Cardiac Health Data Base-Identification of Discriminatory Biomarkers Based on Cardiobioindex

A variety of assay methods are applied here to determine the relative amounts of series of biomarkers in saliva (and/or serum) in healthy and cardiac patients, as classified by the occurrence of AMI. These methods may include, but are not limited to, proteomic chips, Luminex® technology, and lab-on-a-chip (LOC) technologies. Here, saliva (and/or serum) samples obtained from healthy and cardiac patients are tested in parallel by the same method. A cardiobioindex is then determined, reflective of the biomarker(s) contribution to the classification of healthy and cardiac disease status. The cardiobioindex is determined by assigning a relative score for each biomarker based on its signal intensity (or its concentration, after interpolating from a dose response curve with a set of protein standards). A single biomarker index, and/or an aggregate biomarker index based on a set of biomarkers, are then evaluated for their capacity to discriminate between/classify healthy and cardiac patients. Parameters, such as sensitivity (ability to identify a true cardiac patient) and specificity (ability to identify a true healthy patient), and overall accuracy (Ratio of Number of Correct Predictions to Total Number of Patients) of result are determined.

Therefore, the cardiobioindex could be defined by the area under the curve (AUC) fro ROC analysis and describes the sensitivity, specificity and overall accuracy of the test.

By using the cardiobioindex, discriminatory/classification biomarkers for cardiac disease are identified and defined. The accumulation of such information may be used to define threshold values (concentrations) for yes/no tests (such as in the diagnosis of AMI) or quartiles of risk (classification of risk and AMI prognosis) that would eventually be used to classify patients of unknown cardiac status and patients at risk. Here, a cardiac health data-base will be generated based on cardiobioscores, after testing a large number of healthy and cardiac patients at different stages of disease. A sample of unknown cardiac health status may thus be compared for its levels of the same relevant biomarkers against the existing cardiac health cardiobioindex data base, to classify the subject in terms of cardiac health status and relevant risk for future cardiac events. Example 1 below describes a method by which the cardiac health cardiobioindex database can be created.

### V. Use of Saliva as a Diagnostic Fluid

Interest in saliva as a diagnostic medium has increased dramatically during the last decade, as saliva and other oral fluids have been shown to reflect tissue fluid levels of therapeutic, hormonal, immunological, and toxicological molecules. Oral fluids have also been shown to contain bio-markers associated with infectious and neoplastic diseases (Hodinka et al., 1998; Haeckel, 1989; Mandel, 1990; Mandel, 1993a; Schramm et al., 1992). Similarly, the analysis of salivary fluids, like blood-based assays, has the potential to yield useful diagnostic information for the assessment and monitoring of systemic health and disease states, exposure to environmental, occupational, and abusive substances, as well as for the early identification of harmful agents dispersed by bio-terrorist activities (Aguirre et al., 1993).

The major advantages for using saliva in diagnosis relative to blood-based assays have been described in some detail previously (Mandel, 1990; Ferguson, 1987; Mandel, 1993b; Mandel, 1993c; Malamud, 1992; Slavkin, 1998). Saliva collection may be done by procedures that are considered to be non-invasive, painless and convenient. Consequently, these methods may be performed several times a day under circumstances where it may be difficult to collect whole blood specimens.

Many important biological substances including electrolytes (Aps and Martens, 2005; Haeckel and Hanecke, 1993), drugs (Cone, 1993; Jarvis et al., 2000; Svojanovsky et al., 1999; Toennes et al., 2005; Walsh et al., 2003; Zevin et al., 2000), proteins (e.g., cytokines, hormones, enzymes) (Grisius et al., 1997; Hanemaaijer et al., 1998; Lamster et al., 2003; Mogi et al., 1993; Rhodus et al., 2005; Yang et al., 2005), antibodies (Chia et al., 2000; Nogueira et al., 2005; Stroehle et al., 2005), microbes (Stroehle et al., 2005; Lins et al., 2005; Suzuki et al., 2005), and RNAs (Fox et al., 1998; Li et al., 2004a; Li et al., 2004b; St John et al., 2004) have been identified in saliva. Oral fluid presents itself as the ideal diagnostic fluid. There is accumulating evidence that saliva is the “mirror of body”, this makes it a perfect medium to be explored for a non-invasive health and disease monitoring. The translational applications and opportunities are of great potential significance. The ability to classify risk, stratify and monitor health status, disease onset and progression, and treatment outcome monitoring through non-invasive means is a most desirable goal.

A. Association Between Oral Disease and Cad

Historically periodontitis has been considered a disease with ramifications localized to the oral cavity, and in much of the population is viewed as a cosmetic problem, with a permanent solution affected by removal of the teeth, i.e. edentulism. However, recent data support that this chronic infection with continued stimulation of the inflammatory responses of the host communicates with the systemic circulation and may contribute to systemic disease sequelae, such as cardiovascular disease. Indeed, numerous case control and cohort studies have indicated that patients with periodontitis have an increased risk of CVD, i.e., acute myocardial infarction (AMI), stroke and peripheral arterial disease, when compared with subjects with a healthy periodontium.

However, because evidence of the link has come to light only recently, few studies have looked directly at the mechanisms by which periodontitis might contribute to cardiovascular disease. One possibility is that bacteria from the mouth—or products released by these bacteria-travel through the bloodstream to other parts of the body, where they damage the linings of blood vessels. On the one hand, the association between periodontitis and CVD may be linked through common risk factors such as smoking, diabetes mellitus, aging, male gender, and social-economic factors. On the other hand, there is evidence of periodontitis serving an independent risk factor of CVD (DeStefano et al., 1993; Desvarieux et al., 2005; Joshipura et al., 1996; Mattila et al., 1989). Disturbances in the plasma lipoprotein metabolism, systemic inflammatory reactions as well as local inflammation of the artery wall are considered to contribute to the development of early atherosclerotic lesions in CVD (Blake et al., 2003; Ross, 1999).

Recently, it has been shown that periodontitis is often associated with endotoxemia and mild systemic inflammatory reactions, such as an increase in CRP and other acute phase reactants, while periodontal pathogens have been identified in early atherosclerotic lesions (Haraszthy et al., 2000; Noack et al., 2001; Wu et al., 2000). Furthermore, several groups have reported elevated serum CRP levels in periodontitis patients. The extent of increase in serum CRP levels in periodontitis patients correlates significantly with the severity of the disease, even with adjustments for smoking habits, body mass index, triglycerides, and cholesterol levels. Interestingly, there seems to be an indirect association between the occurrence of periodontal conditions and an increased risk for CVD. The positive correlation between CRP and periodontitis may indicate that circulating inflammatory molecules contribute to the pathogenesis of both conditions and studies that determine the level of CRP, and other inflammation markers, in the fluids of the oral cavity could help us better understand the relationship of these two inflammatory diseases (Noack et al., 2001; Loesche, 1994).

B. Utility of Salivary Diagnostics for Systemic Diseases

In the past, only but a few studies targeted the use of saliva as a diagnostic fluid for systemic diseases. Impediments to the use of oral fluids have been the relatively low concentration of various important biomolecules in saliva, in comparison to serum or plasma, accompanied by a lack of sufficiently sensitive assays and equipment that could be used in dental healthcare settings (Kaufman and Lamster, 2004). Therefore, up to until now, it remained unclear what salivary analyte targets could be useful as adjunctive clinical information for a systemic disease, such as CVD. Clearly, studies have been needed that define these relationships before the diagnostic utility of saliva could be promoted.

Modern analytical technologies are expected to extend vastly the potential diagnostic value of oral fluids. To be useful, salivary biomarkers must be accurate, biologically relevant, discriminatory, and at measurable concentrations. The identification of these biomarkers for chronic inflammatory diseases, including cardiovascular disease, from the array of potential markers, promises to create a quantum leap in cardiac diagnostics.

### VI. Generation of Classification Algorithms for Qualifying CVD Status

In certain embodiment, a detection device can comprise any device or use any technique that is able to detect the presence and/or level of a biomarker in a sample. Examples of detection techniques that can be used in a detection device include, but are not limited to, Lab-on-a-chip (LOC), nuclear magnetic resonance (NMR) spectroscopy, 2-D PAGE technology, Western blot technology, immunoanalysis technology such as ELISA, electrochemical detectors, spectroscopic detectors, luminescent detectors, microarray, and mass spectrometry. The output from a detection device can be processed, stored, and further analyzed or assayed using a bio-informatics or a computer system. A bio-informatics system can include one or more of the following: a computer; a plurality of computers connected to a network; a signal processing tool(s); and a algorithm.

In some embodiments, data derived from the detection device that are generated using samples such as “known samples” can then be used to “train” a classification model. A “known sample” is a sample that has been pre-classified. The data that are derived from the detection device and are used to form the classification model that can be referred to as a “training data set.” In accordance with the certain aspects of the present invention, the training data set will comprise data on CBI of biomarkers and their threshold concentrations. And the algorithm comprised in the bio-informatics system may be used to calculate the CBI score and establish the threshold concentration for classification as quartiles for risk or presence/absence (yes or no tests) based on the methods of the present invention. Once trained, the classification model can recognize patterns in data derived from the detection device generated using unknown samples. The classification model can then be used to classify the unknown samples into classes. This can be useful, for example, in predicting whether or not a particular biological sample is associated with a certain biological condition (e.g., diseased versus non-diseased), in diagnosis or prognosis of certain cardiovascular diseases, or in classifying risk level for cardiovascular diseases.

The training data set that is used to form the classification model may comprise raw data or pre-processed data. In some embodiments, raw data can be obtained directly from a detection device, and then may be optionally pre-processed.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. Patent Application No. 2002 0138208.

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580, U.S. Patent Application No. 2002 0193950, U.S. Patent Application No. 2003 0004402, and U.S. Patent Application No. 2003 0055615.

The classification models can be formed on and used on any suitable digital computer. Suitable digital computers include micro, mini, or large computers using any standard or specialized operating system, such as a Unix, Windows™, or Linux™ based operating system. The digital computer that is used may be physically separate from the detection device that is used to create the data of interest, or it may be coupled to the detection device.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for CVD. The classification algorithms, in turn, form the base for diagnostic tests by providing diagnostic values (e.g., cut-off points or threshold levels as well as CBI or CBScore) for biomarkers used singly or in combination.

### VII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Creation of a Cardiac Health Cardiobioindex Database

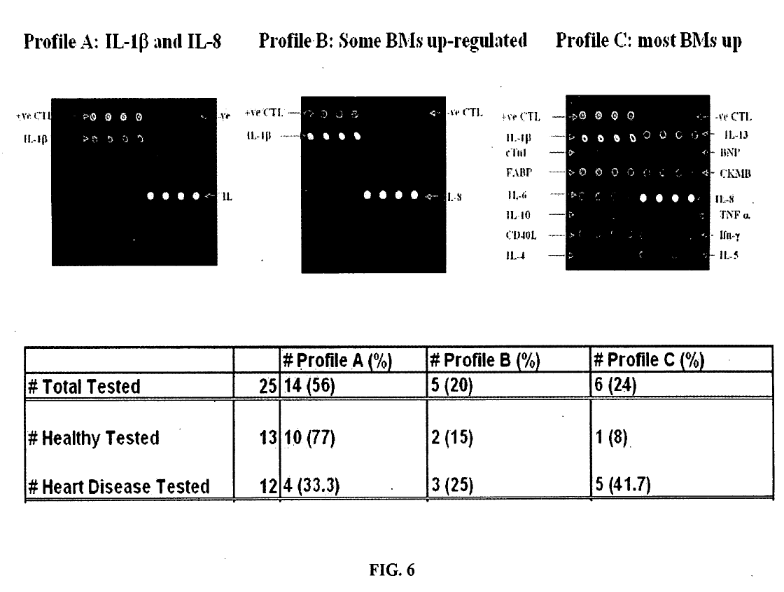

In this study, saliva samples obtained from healthy individuals (n=13) and cardiac patients (n=12), were tested by a cardiac proteomic chip targeting the following cardiac biomarkers: IL-1β, IL-13, cTnI, BNP, FABP, CKMB, IL-6, IL-8, IL-10, TNF-α, CD40L, IFN-γ, IL-4 and IL-5 (FIG. 6).

Results were evaluated in terms of the biomarker profile of the array. Three biomarker profiles were identified. The first, profile A, shows detection of two biomarkers (IL-1β and IL-8); the second, profile B, shows up-regulation of IL-1β and IL-8, as well as some of the other biomarkers in the array. The third, profile C demonstrates up-regulation of all BMs evaluated. Results show that the majority (77%) of the healthy patients exhibit Profile A, while 42% of cardiac patients show a response consistent with Profile C. A small percentage from the two groups exhibit a cardiac array consistent with Profile B, a profile that may be characteristic of apparently healthy individuals at risk for developing cardiac disease.

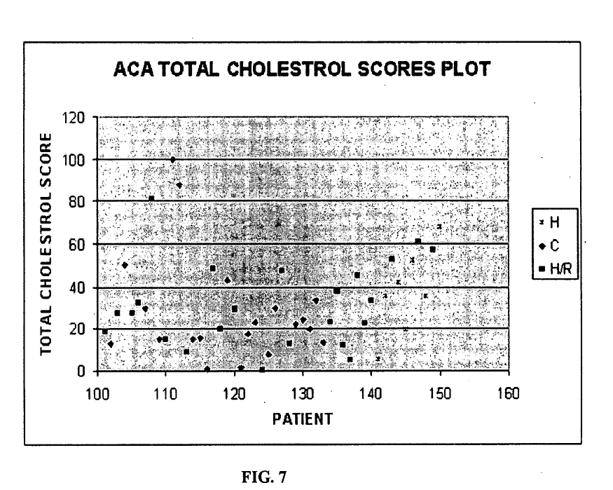

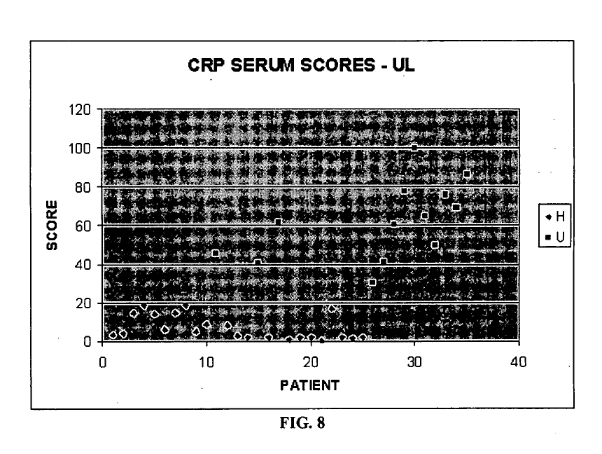

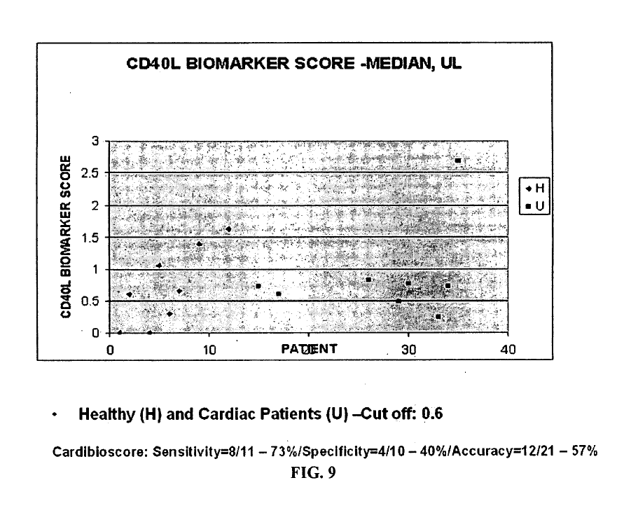

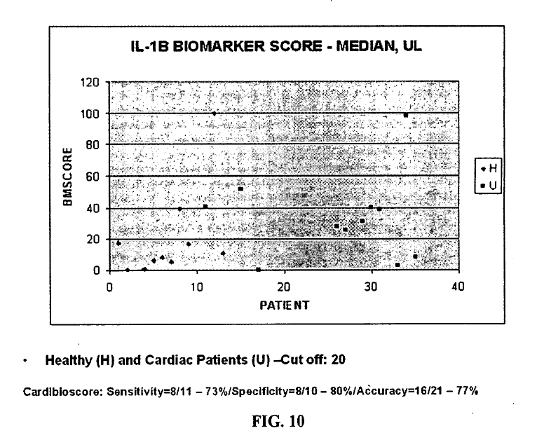

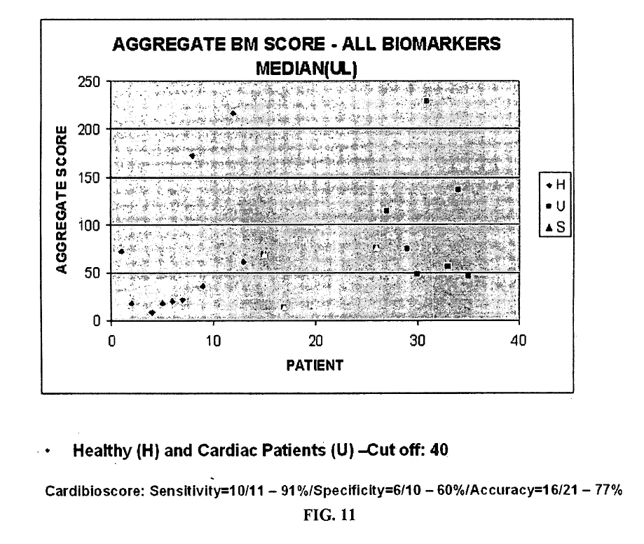

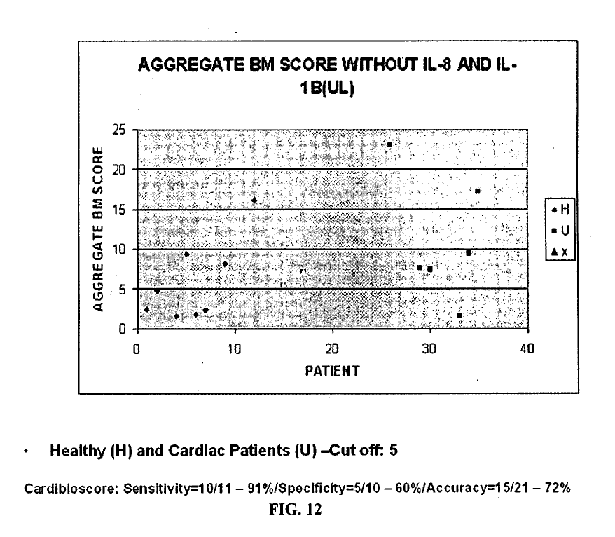

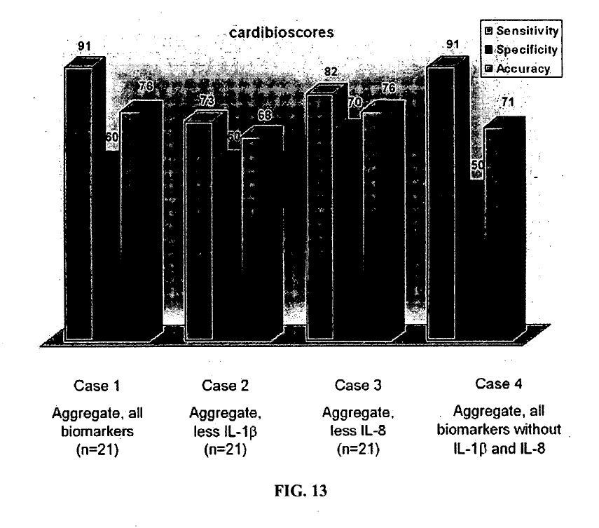

FIGS. 7-13 show the initial approach of analyzing biomarker data on cardiac biomarkers. Having realized that the above approach of evaluating cardiac array results is qualitative and, thus, limiting, the inventors developed the following method by which cardiac biomarkers in the array were assigned an index (cardiobioindex) for their ability to classify healthy individuals and cardiac disease patients. This methodology has the advantage that the contributions of the biomarkers in cardiac health assessment are weighted. Thus, the relative attributes of the individual biomarkers can be assessed as well as the utility of the various combinations. Further, the scores are normalized so that the biomarker concentration range can be accounted for.

The following procedure is followed to derive the single or aggregate biomarker cardibioscore:
a. The protein levels for all healthy controls and CAD case samples are measured and results were recorded.
b. The relative levels of each biomarker are normalized for all subjects and a dichotomous approach is used to divide the sample population into two well-phenotyped, “control” and “CAD”, populations
c. Logistic regression models are used for the analysis of data. The logistic regression model intrinsically attributes different weights for each of the biomarkers. Statistica 5.5 software platform was used for the logistic regression, with the maximum likelihood as the loss function. The method chosen for the estimation was a Hooke-Jeeves pattern moves, with a maximum number of iterations set at 50 and a convergence criterion of 0.0001.
d. Values of the area under the curve (AUC), or C statistic are computed, as well as the standard error (SE), and applied using a two tailed p-value at the 95% confidence level. The biomarker utility index, or cardiobioindex, for each biomarker or combination (panel) of biomarkers was defined simply by the AUC or the C-statistic.
e. Fourth, the sensitivity and specificity for single biomarker and biomarker aggregates are measured. Here, the best ROC curve from a variety of inputs (biomarkers) is used along with definition of the beta weights to create an index that can be used to classify the patients. The predicted values are used to construct ROC curves of the total positive response (TPR) as a function of false positive rate (FPR), using analyse-it (Analyse It Software, Ltd).

### Example 2

### Assignment Of Cardiovascular Biomarker Index Or Cardiobioindex

### I. METHODS

Patient Recruitment, Sample Collection, and Testing. Patients were recruited at 3 clinical sites: Austin Cardiovascular Associates (ACA) in Austin Tex., the University of Louisville (UL), Louisville, Ky. and University of Kentucky (UK) in Lexington, Ky. Institutional Review Board approval was obtained at each center.

Thirty-five subjects, 22 who had no known CVD and 13 with verifiable heart disease (CAD, cardiomyopathy, microvascular angina, hypertension, CHF) participated in the UL study. All study participants provided saliva in 50 mL sterile, plastic specimen tubes. Subjects rinsed their mouth with water before sample collection so as to remove any foreign matter that may be present. Unstimulated saliva was then expectorated into the specimen tube until a total of approximately 2 mL per subject was obtained. Samples were positioned upright in a Styrofoam test tube holder in a cooler that contained dry ice and then transferred to storage at −70° C. until shipment to UK. After sample collection, a nurse or other trained personnel collected the requisite medical information.

Thirteen subjects, 4 healthy and 9 AMI [3 ST elevation myocardial infarction (STEMI) and 6 non-ST elevated myocardial infarction (NSTEMI)] patients participated in the UK study. Unstimulated saliva samples were collected within 48 hr of the AMI, aliquoted into 1 mL tubes, and stored at −70° C. Aliquots of samples collected at UK were tested locally using the Luminex®—based multiplexing or ELISA approaches. Duplicate aliquots of the same samples were shipped frozen on dry ice to the University of Texas at Austin for analysis with proteomic μ-array chip and LOC system.

Twenty-nine subjects over 21 years of age, 20 who had been diagnosed with coronary artery disease (CAD) and 9 healthy controls, were recruited from the ACA site. All study participants were asked to complete a questionnaire that recorded the age, gender, smoking status, exercise frequency, weight and height (for BMI calculation), information regarding their own, and their family's medical history with respect to CVD, cancer, and a number of inflammatory conditions. Each subject donated 5-10 mL of whole blood. Approximately 2-4 mL of serum was retrieved from the coagulated blood and divided in two aliquots before freezing. The first aliquot was transported on ice to a local pathology laboratory (Clinical Pathology Laboratories-CPL, Austin, Tex.) for lipid analysis. The second serum aliquot was transported on ice to UT for CRP measurements using the LOC assay platform.

LOC for the measurement of CRP. Previous studies have described the design, fabrication and testing of nano-bio-chip structures whereby immunoassays were performed on chemically sensitized beads that were arranged in an array of wells etched on silicon wafers with integrated fluid handling and optical detection capabilities (Christodoulides et al., 2002; Christodoulides et al., 2005b, each of which is incorporated herein by reference in its entirety All the experiments described in this study utilized agarose bead sensors developed in the McDevitt laboratories.

In this study, a sandwich-type immunoassay was used for the measurement of the biomarker CRP using the LOC system. Beads coated with a capturing antibody (Accurate Chemical, Westbury, N.Y.) for CRP were sequentially exposed to the analyte protein standard (Cortex Biochemicals, San Leandro, Calif.) or the unknown sample and to a detecting antibody (Accurate Chemical, Westbury, N.Y.) conjugated to Alexafluor-488 to produce a CRP/dose-dependent fluorescent signal within and around the bead. The top insert of the flow cell allowed for the microscopic evaluation of signals generated within the array, which were subsequently captured by a charge-coupled device (CCD) video chip along with the use of transfer optics. Here, after each assay run, the final image of the bead array was captured with the CCD, digitally processed and analyzed, and the signal intensity converted for each bead into a quantitative measurement based on the generated standard curve. Likewise, digital information from each array/trial was obtained using Image Pro Plus software and analyzed with SigmaPlot®. The concentration of the unknown sample was extrapolated from the generated standard curve. The data was analyzed using a four parameter logistic equation process within the SigmaPlot® environment to generate a standard, dose-response curve and to predict concentrations of the unknowns.

ELISA testing. Samples were tested for CRP using a clinically-validated high sensitivity (hs)CRP ELISA kit obtained from ALPCO (Windham, N.H.). Commercial ELISA kits were also used for ENA-78 (R&D Systems, Minneapolis, Minn.), IL-18 (Medical & Biological Laboratories Co, Naka-ku, Nagoya, Japan), TnI (Life Diagnostic, West Chester, Pa.), and CD31/PCAM-1, sICAM-2, sICAM-3, sVCAM-1 (Diaclone BESANCON Cedex, France). The concentration values from the ELISA studies were determined using a Molecular Devices SpectraMax M2 (Sunnyvale, Calif.) and data analysis software SOFTmax PRO.

μ-array measurements. Allied Biotech's (Ijamsville, Md.) antibody-based human cardiovascular micro-array kit, designed to screen diverse biological samples, such as cell lysates, serum, plasma, and tissue culture supernatants, was used in this study to test for the presence of 14 different cardiovascular markers TNFα, IL-4, INF-γ, sCD-40L, BNP, FABP, cTnI, CKMB, IL-1α, IL-5, IL-6, IL-8, IL-10 and IL-13 in saliva. Each slide in the kit contained 16 identical arrays of 14 capture antibodies in quadruplicate and supported the analysis of up to sixteen 40-μL samples. A cocktail of biotinylated detection antibodies combined with the capturing antibodies spotted on the slide, comprised the antibody pairs to detect the biomarkers. Biomarker detection was achieved with the addition of Streptavidin-Cy5 conjugate, for a fluorescent-based detection. Positive and negative controls spotted within each array allowed for assay validation. A μ-array scanner (GenePix Personal 4100A, Molecular Devices Corporation, Sunnyvale, Calif.) was used, in conjunction with compatible image analysis software (GenePix Pro 6.0, Molecular Devices Corporation, Sunnyvale, Calif.), to determine the background-subtracted signal of each spot. The quadruplicates were then averaged to quantify the specific signal to noise ratio for each biomarker on the array. Using the average signal intensity of a negative control sample (3% BSA/PBS) as a baseline, allowed determination of the relative abundance of each biomarker in each sample. For some, but not all biomarkers, the concentration of each analyte in the samples was determined using a standard curve.

Luminex®. Multiplexing beadlyte technology using a Luminex IS-100 instrument (Luminex Corp., Austin, Tex.) was employed for a number of the analytes. Reagent kits for IL-1β, IL-6, MCP-1, RANTES, and TNFα were obtained from Upstate Co. (Temecula, Calif.). For CRP, leptin, adiponectin, E-selectin, MMP-9, MPO, sICAM-1, sVCAM-1, fractalkine, and sCD40L the kits were acquired from Linco Research (St. Charles, Mo.). All assessments were according to the manufacturer's instructions with the exception of the Upstate panel of analytes. This panel was modified to increase sensitivity by approximately 5-fold over the standard procedure supplied with the commercial kits.

Lipid measurements. TC and HDL were measured enzymatically using a Hitachi 911 autoanalyzer (Roche Diagnostics, Basel, Switzerland), and LDL was directly measured in a CLIA-certified lab (Genzyme, Cambridge, Mass.).

Statistics and construction of ROC curve. For the analysis of the combination of biomarkers (IL-1β, IL-13, BNP, IL-6, TNF-α, IL-10, IL-4, sCD40L, IL-8, IL-5, and CRP), both linear and logistic regression models were used. In the case of the linear regression, all weights were assumed to be the same allowing for the simple addition of the biomarker contributions. For the protein array data, the average of the median spot intensity was calculated for each biomarker, and served as an independent variable in the analysis. For the LOC and Luminex® data, the concentration of the biomarkers was extracted based on a 4-parameter logistic curve using SigmaPlot®, and served as the independent variable in the logistic regression. Statistica 5.5 software platform was used for the logistic regression, with the maximum likelihood as the loss function. The method chosen for the estimation was a Hooke-Jeeves pattern moves, with a maximum number of iterations set at 50 and a convergence criterion of 0.0001.

The predicted values were then used to construct ROC curves of the total positive response (TPR) as a function of false positive rate (FPR), using analyse-it (Analyse It Software, Ltd). The TPR determines the performance of a biomarker, or of a collection of biomarkers, on classifying cardiac patients correctly among all cardiac samples available in this study. The FPR, on the other hand, defines how many incorrect samples are identified as cardiac, while they are actually healthy, among all healthy samples available during the test. The ROC space is defined by FPR and TPR as x and y axes respectively, and depicts relative trade-offs between true positive (benefits) and false positive (costs). The best possible prediction method would yield a point in the upper left corner or coordinate (0,1) of the ROC space, representing 100% sensitivity (all true positives are found) and 100% specificity (no false positives are found). The (0,1) point would also be associated with perfect classification capabilities. Alternatively, a completely random guess would give a point along a diagonal line (the so-called line of no-discrimination) from the left bottom to the top right corners. The diagonal line, thus, determines the areas that indicate good or bad classification/diagnostic results. Points above the diagonal line indicate good classification results, while points below the line indicate poor classification capabilities. Values of the area under the curve (AUC), or C statistic were computed, as well as the standard error (SE), and applied using a two-tailed p-value at the 95% confidence level.

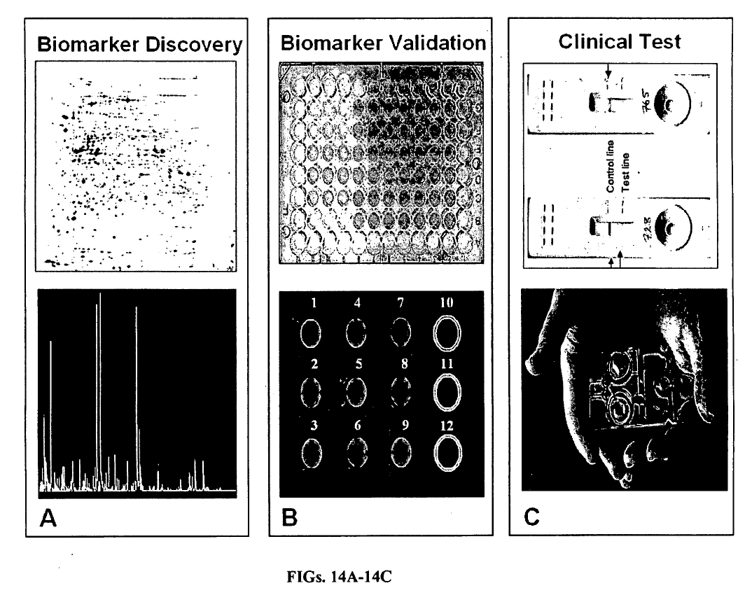

Evaluation of protein biomarkers associated with CVD. In an effort to create more powerful risk prediction and biomarker validation tools, the inventors evaluated established and novel protein biomarkers that have been associated with CVD. Clearly, the discovery of new biomarkers represents only the initial step that is required to develop and secure approval for new diagnostic tests (FIGS. 14A-14C). (Anderson, 2005a; Anderson and Anderson, 2002; Ludwig, and Weinstein., 2005; Omenn, 2006; Zolg, 2006; Hortin et al., 2006; Anderson, 2005b). Four high throughput proteomic methods were applied to quantify cardiovascular biomarkers: (1) ELISA, (2) Luminex® liquid array methodologies, (3) protein μ-arrays and (4) the electronic taste chip method (i.e., the bead-based lab-on-a-chip system). These four methodologies provide the capacity to acquire data that can define biomarker performance based on sensitivity, selectivity, and accuracy, and can be validated in well-phenotyped populations. Following the validation step, where the targets are identified and assay expectations are defined, it is often necessary and desirable to move the biomarker assay into a format that is more suitable for clinical use. It is in this capacity that both lab-based instruments as well as point-of-care devices can be envisioned for the cardiac diagnostics area and both have precedent in modern clinical settings (Sluss, 2006; Wu, 2006).

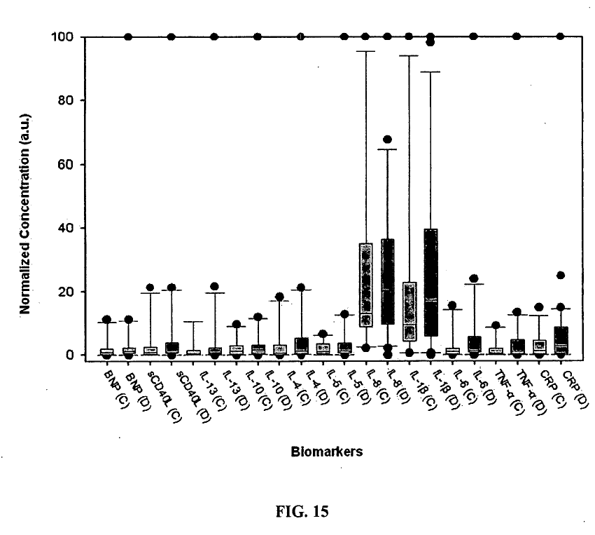

Three case-control studies were concurrently conducted to demonstrate the new biomarker validation procedures. In this effort, ELISA, protein μ-array, Luminex®, and bead-based LOC approaches were applied to achieve the detection of 28 different biomarkers of CVD in the serum and saliva of control and cardiac disease patients. Representative data obtained in one of these studies with one of the fore-mentioned approaches, the protein μ-array, are provided in FIG. 15. Here, the relative concentration range of a number of biomarkers, as well as the median concentrations for the two populations, as indicated by the relative fluorescence intensity of the spots in the μ-array, for control and diseased patient groups, are plotted. From these data those achieved from the ELISA, Luminex®, and LOC measurements, it is clear that there is a significant overlap between the healthy and control biomarker concentration ranges. As such, this approach of evaluating single biomarkers does not provide classification capabilities that may be viewed as desirable for the new biomarkers. Nonetheless, upon closer examination of all accumulated data, it can be found that certain expression patterns do indeed exist suggesting that biomarker combinations may be more revealing. This exciting finding is found to be consistent across patient groups from all three clinical sites studied, as described in more detail below. However, in order to extract these patterns, it is essential that clearly defined analytical procedures of expression profiles are made rather than single biomarker comparisons.

Cardiobioindex method. To promote a better evaluation of the biomarker capability to discriminate between control and diseased populations, the inventors developed a simple, yet novel, scoring system by which a single and/or an aggregate (based on a set of biomarkers) biomarker score can be determined. This method assigns a numerical index, defined here as a CARDIOvascular BIOmarker INDEX or cardiobioindex, to each, and/or a combination, of biomarkers, as measured by a variety of detection/measurement methods. The index serves to quantify the effectiveness of these biomarkers to classify patients that may or may not have CVD. The cardiobioindex is derived from the area under the receiver operating characteristic (ROC) curve as applied to the classification of coronary artery disease (CAD), STEMI, NSTEMI, cardiomyopathy, microvascular angina, hypertension, and chronic heart failure (CHF). According to this method, the best ROC curve from a variety of inputs (biomarkers) is used along with definition of the beta weights to create an index that can be used to classify the patients. This allows the integration of several biomarkers into a clinically useful schema for patient classification. This index, thus, serves as a vehicle to secure valuable insight into the performance potential for various single biomarkers and biomarker combinations. The cardiobioindex is a reflection of the overall accuracy of the salivary/serum biomarker(s) evaluated for classifying control and cardiac patients.

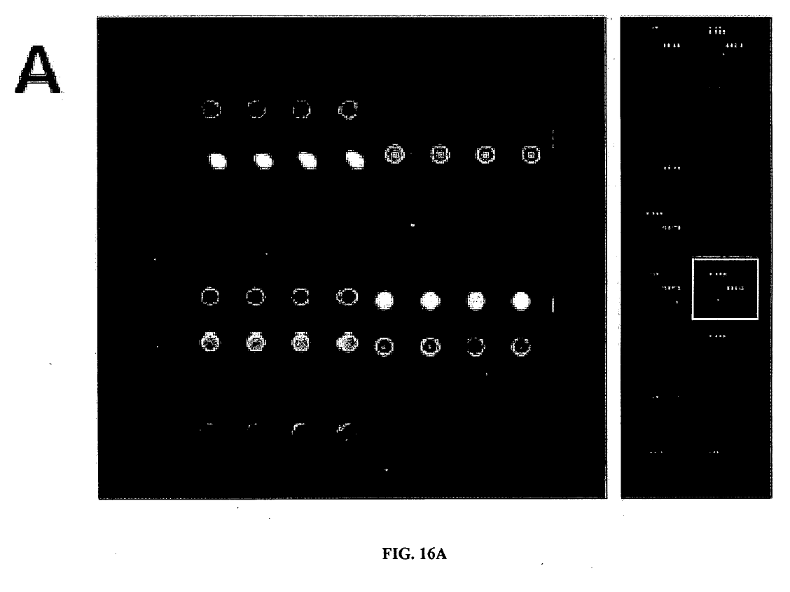

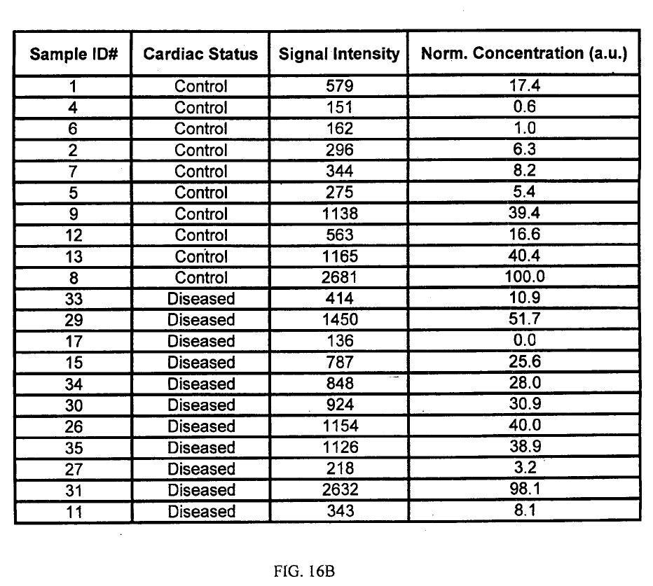

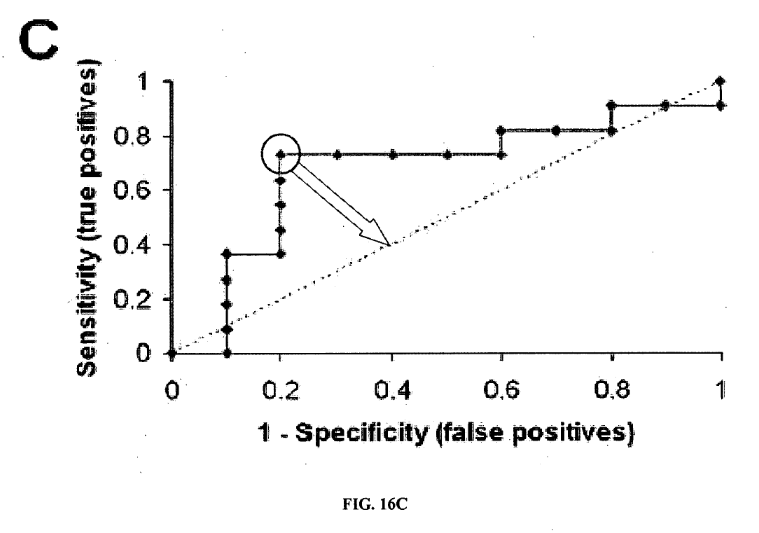

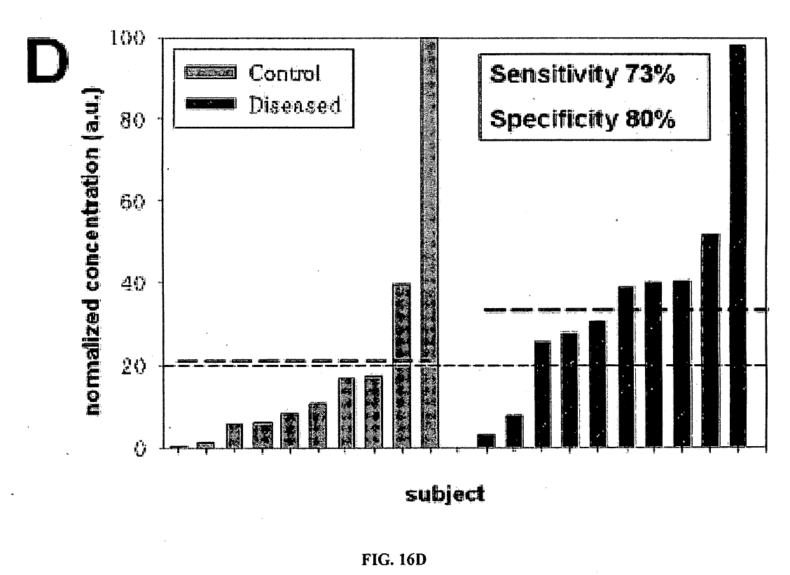

The mechanics for the development of the cardiobioindex are depicted in FIGS. 16A-16D. Four main steps are used to decipher the index. First, the concentration levels for all control and case (i.e., CVD) samples are collected for all biomarkers of interest and the results are recorded. If the assay is semi-quantitative (as is the case for many μ-array approaches), the relative signal intensities are used to record differences in biomarker levels between samples. If the assay is quantitative, biomarker concentrations interpolated from dose response curves are used to record differences in biomarker levels between samples. Second, a dichotomous approach is used to divide the sample population into “control” and “diseased” populations, the latter encompassing the various sub-categories of cardiovascular disease. A logistics regression model is used here at it allows the manipulation of dichotomous data as required for patient classification. Third, the cardiobioindex is extracted from the area under the ROC curve, or the C-statistic, for each biomarker, or for a combination of biomarkers. Values of the C statistic range between 0.5 and 1.0, and a value closer to 0.5 indicates that the model lacks predictive power, and a value closer to 1.0 demonstrates the model's ability to assigning higher probabilities to correct cases. Forth, patients are ranked with respect to their cardiobioindex values for both the diseased and control populations. With this ranked evaluation of the patients, it is possible to select threshold values and to calculate the sensitivity and specificity for this particular biomarker index.

Validation of the cardiobioindex method. This cardiobioindex method is validated here for its capacity to correctly classify healthy and cardiac patients within the context of the three pilot studies, performed in parallel with collaborators at the ACA, UK and UL sites (See Methods Section for more details). Physical parameters (i.e., BMI, blood pressure), established serum risk factors (total cholesterol, HDL, LDL, lipid ratios), as well as a series of novel and emerging biomarkers of CVD disease, as measured in the saliva of control and cardiac patients, are evaluated as, single or aggregate, classifiers of cardiac disease.

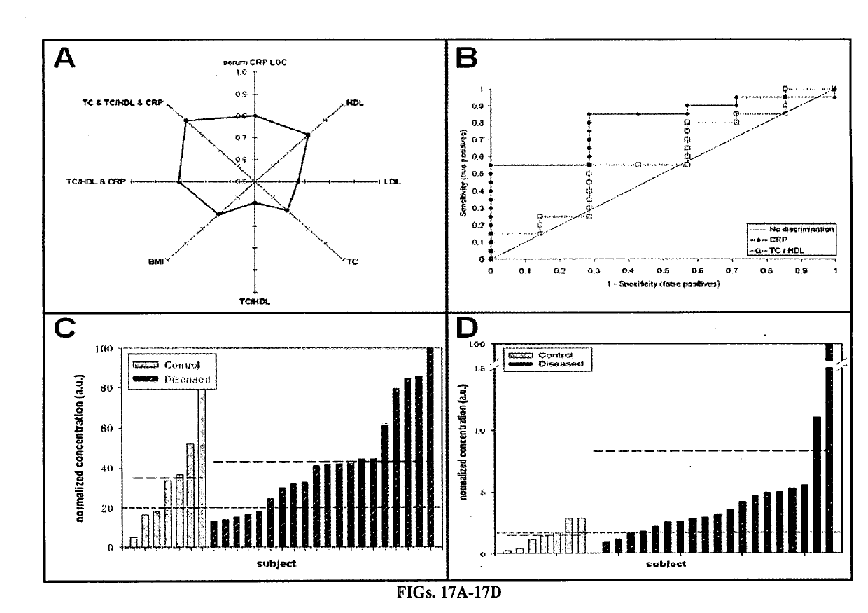

In the initial pilot study (ACA), the new biomarker scoring method is first validated within the context of the most accepted/established risk factors that are currently in place for ischemic/atherosclerotic CVD. Accordingly, established biomarkers of CVD that include TC, HDL, LDL, CRP, and their various combinations, are first evaluated in serum and scored. The cardiobioindex for the physical parameter BMI is also evaluated and compared to the cardiobioindices measured for the serum biomarkers. These control studies allow for an establishment of the baseline performance index for these traditional risk factors that can be used later to evaluate the relative classification capabilities of the novel biomarker panels, as well as the utility of the novel biofluid matrix, saliva. Likewise, serum cholesterol and serum CRP measurements (performed by a clinical laboratory and by LOC, respectively) for 27 samples (7 control and 20 cardiac disease patients) are used to generate the first cardiobioindex for these established cardiac risk factors. Classifiers of cardiac disease with varying input values, or cardiobioindices, as defined by the C-statistic or the equivalent area under the ROC curve, are identified. Importantly, this index method identifies all these parameters as useful classifiers of cardiac disease (FIG. 17A).

From these biomarker inputs and the various biomarker combinations, TC, TC/HDL and LDL performed the poorest, with cardiobioindices of 0.682, 0.593 and 0.671, respectively. The cardiobioindex for BMI secured a value of 0.707, while CRP, alone or in combination with TC/HDL as well as TC plus TC/HDL achieved superior cardiobioindices values of 0.8 (SE 0.0894, p-value 0.0004 and 95% confidence interval (CI): 0.625-0.975), 0.807 (SE 0.1016, p-value 0.0013 and 95% CI:1 0.608-1.000) and 0.893 (SE 0.0609, p-value, 0.0001 and 95% CI: 0.774-1.000), respectively (FIG. 17A and FIG. 17B). It is interesting to note that the cardiobioindices for the CRP and TC/HDL inputs in classifying control and cardiac patients is consistent with the reported relative value of the two biomarkers as risk factors for the development of arteriosclerosis and CVD (FIG. 17C and FIG. 17D) (Rifai and Ridker, 2003; Ridker et al., 2002). This agreement with the prior literature serves to provide some confidence and validation of the new methodology.

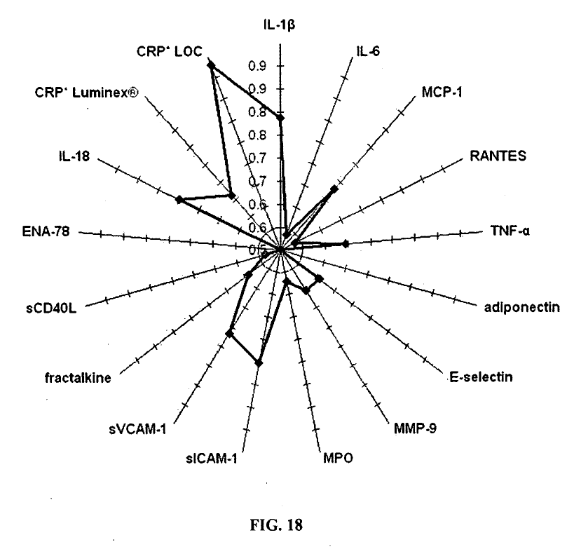

Cardiobioindex values of biomarkers in saliva. The performance for a set of individual CVD biomarkers was further studied within the context of saliva measurements. Biomarkers IL-1β, IL-6, MCP-1, RANTES, TNF-α, adiponectin, E-selectin, MMP-9, MPO, sICAM-1, sVCAM-1, fractalkine, sCD40L, ENA 78, IL-18 and CRP are measured in the saliva of control and cardiac disease patients. FIG. 18 provides a summary of the data from the comparison of the individual biomarkers. The classification capability for cardiac disease for varying input values is assessed. The cardiobioindex values for biomarkers RANTES, ENA 78, fractalkine, adiponectin, sCD40L, MPO, MMP-9, E-Selectin and IL-6 were found to be 0.6 or lower, suggesting these biomarkers offer rather poor discrimination capabilities, while other inputs, such as IL-1β, sICAM-1, TNF-α, sVCAM-1, MCP-1, CRP and IL-18 demonstrated good to excellent discrimination utility with cardiobioindex values ranging from 0.65-0.929. It should be noted that the apparent poor performance demonstrated by some of these emerging biomarkers of CVD could be a result of inefficiencies associated with the method employed for their measurement. A less sensitive analytical method is not expected to be able to detect, and, thus, measure accurately the less abundant proteins in the complex fluid of saliva. In contrast, an assay with enhanced detection capabilities can detect the analyte/biomarker in a more sensitive manner and, thus, detect differences of the biomarker levels between control and disease groups, for a more reliable biomarker validation effort.

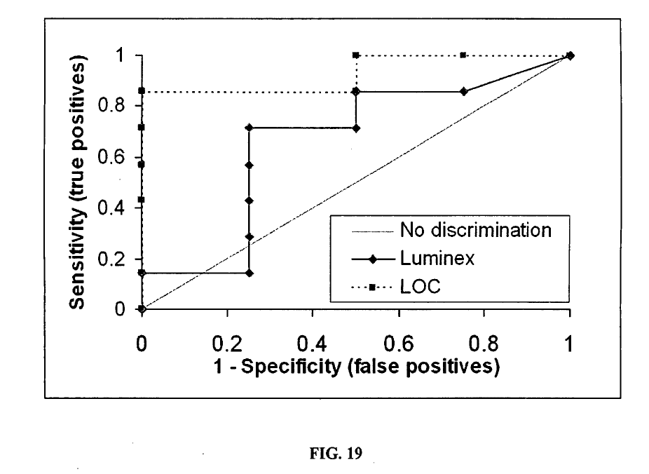

When the same samples are tested for salivary CRP by LOC and LUMINEX® approaches, a significantly-improved cardiobioindex for the biomarker CRP is achieved with the more sensitive LOC method than with Luminex®) (FIG. 19). Similarly, when conventional clinical lab-based high sensitivity CRP ELISA methods are employed, only 23% of the samples are above the limit of detection of this “high sensitivity” method. Salivary CRP, when measured with the Luminex® system (LOD of 80 pg/mL) demonstrates 71.4 and 75% sensitivity and specificity, respectively, while when measured with the LOC method, CRP correctly classified control from disease patients with 85.7 sensitivity and 100% specificity. Likewise, the Luminex® approach provides a cardiobioindex for CRP of 0.661 (SE 0.1888, p-value 0.1973 and 95% CI: 0.291-1.000), while the counterpart LOC method achieves a cardiobioindex of 0.929 (SE 0.0821, p-value <0.0001 and 95% CI: 0.768-1.000). Indeed, the LOC-based method demonstrates more sensitive and more precise CRP measurements than any of the other established mature technologies (Table 1), many of which are in clinical use as previously noted (Christodoulides et al., 2005b).

TABLE 1
Comparison of assay performance characteristics for various methods of measurement of CRP

| | | | | Limit of | | Intra-Assay | Inter-Assay |
|---|---|---|---|---|---|---|---|
| Organization | Methodology | Useful Assay Range | | Detection | | % CV | % CV |
| UT ETC | LOC (PBS) | 20 fg/mL-100,000 | ng/mL | 10.0 | fg/mL | 8 | 3.0-10.0 |
| | LOC (saliva) | 10-10,000 | pg/mL (1:1000 dilution) | 1.0 | pg/mL | N/A | N/A |
| | LOC (serum) | 0.2-100,000 | ng/mL | 0.1 | ng/mL | N/A | N/A |
| LUMINEX | MPLA | 0.08-250 | ng/mL | 6.0* | pg/mL | 8 | 17.5 |
| Allied Biotech | μ-array | 100-50.000 | pg/mL | 10.0 | pg/mL | N/A | N/A |
| ALPCO | ELISA | 1.9-150 | ng/mL | 0.1 | ng/mL | 6 | 12 |
| Diagnostic Systems | ELISA | 10-500 | ng/mL | 1.6 | ng/mL | 3 | 5 |
| Laboratories | | | | | | | |
| Dade Behring | IN | 175-11,000 | ng/mL | 20.0 | ng/mL | N/A | 4.3-6.8 |
| Wake | IT | 50-10,000 | ng/mL | 60.0 | ng/mL | N/A | 1.0-11.0 |
| Roche | PE-IT | 100-20,000 | ng/mL | 210.0 | ng/mL | N/A | 0.6-7.2 |
| Abbott | MPC | 50-30.000 | ng/mL (1:50 dilution) | | N/A | N/A | 6.7-12.0 |
| Diagnostic Products | IL | 100-250,000 | ng/mL (1.100 dilution) | 20.0 | ng/mL | N/A | 6.4-12.0 |
| Corporation | | | | | | | |
| Beckman Coulter | IN | 1.000-960.000 | ng/mL | | N/A | N/A | 4.0-24.0 |
| Iatron | IT | 50-4,000 | ng/mL | 5.0 | ng/mL | N/A | 1.1-3.4 |
| Daiichi | IT | 200-60,000 | ng/mL | 40.0 | ng/mL | N/A | 1.3-6.1 |
| Denka | IT | 50-10,000 | ng/mL | 30.0 | ng/mL | N/A | 2.2-5.1 |
| Kamiya | IT | 300-20,000 | ng/mL | 100.0 | ng/mL | N/A | 1.51-13.0 |
| Olympus | IT | 500-20.000 | ng/mL | 80.0 | ng/mL | N/A | 3.2-44.0 |

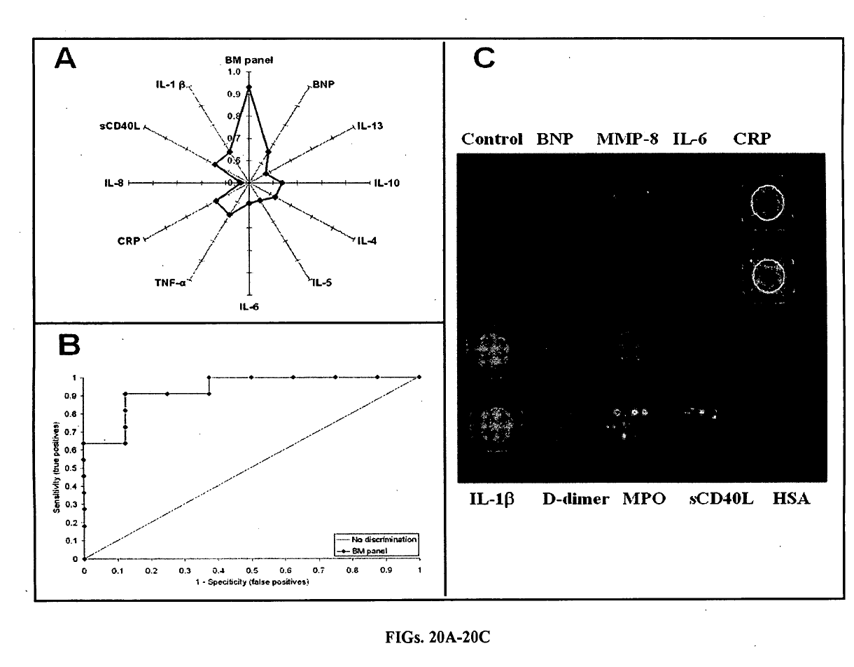

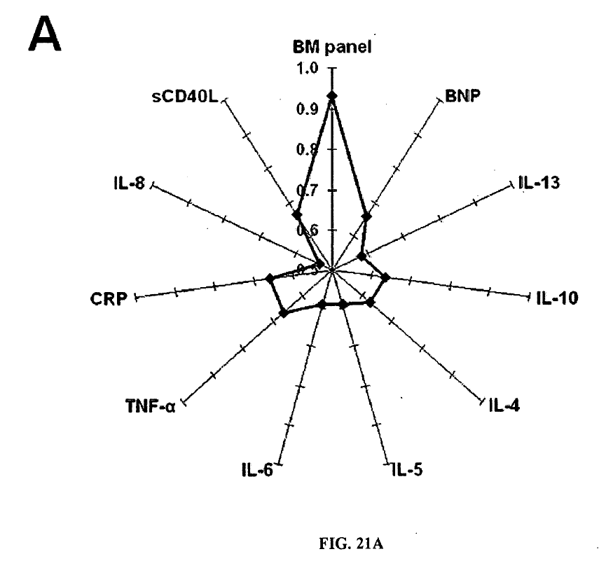

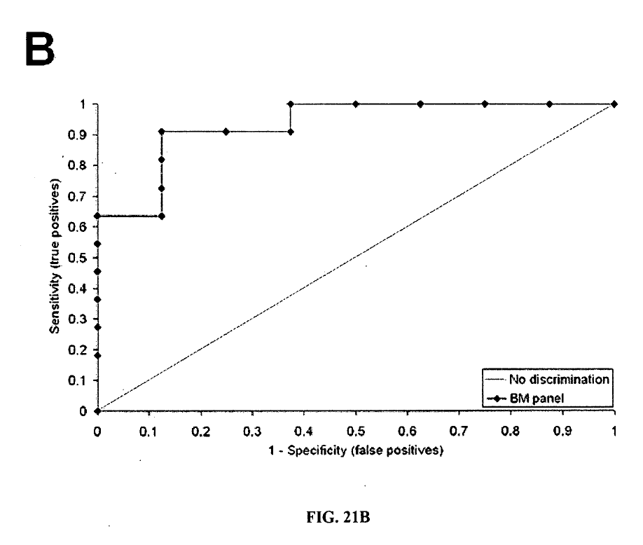

Aggregated cardiobioindex values. In an effort to create more powerful risk prediction and biomarker validation tools, the inventors considered established as well as novel protein biomarkers associated with CVD. It is the working hypothesis that in order to develop accurate biomarker models, it is necessary to consider the global biomarker expression profiles, whereas individual biomarkers only provide select information as related to the various specific stages of CVD. To test this hypothesis, the inventors compared the cardiobioindex values for single biomarkers with those achieved when the same biomarkers are considered in aggregate (FIG. 20A).

For the logistic regression approach, the predicted values of disease prediction (status) is achieved through the use of the following equation (Michel et al., 2003):

$$status = {e}^{( {β}_{0} + {\sum }_{n} {β}_{n} {X}_{n} )} / ( 1 + {e}^{( {β}_{0} + {\sum }_{n} {β}_{n} {X}_{n} )} )$$

where β₀ is the constant of the logistic equation, β₁₋ₙ the weights affecting each biomarker X₁₋ₙ. Single biomarkers IL-1β, IL-13, BNP, IL-6, TNF-α, IL-10, IL-4, sCD40L, IL-8 and IL-5 (as measured by proteomic μ-array chip) and CRP (as measured by LOC) produced cardiobioindices in the range of 0.534-0.665, while their combination, as reflected by the biomarker panel, resulted in a significantly improved cardiobioindex of 0.932 (SE 0.0574, p-value <0.001 and 95% CI: 0.819-1.000) as shown in FIG. 20B. Here, the combination of all of the fore-mentioned biomarkers contributes to the identification of a superior cardiobioindex and allows for the classification of control and cardiac disease patients with 91% sensitivity and 80% specificity. These values, as derived from multiplexed saliva analysis, are considered to be excellent for classifying patients with ischemic heart disease.

In addition to the logistic regression, the inventors also explored the C statistic values obtained from a linear regression model. For the same biomarker panel described above, a value of 0.852, with a standard error of 0.0870 (p<0.0001, CI: 0.682-1.000), is obtained. The inventors hypothesize that the improved statistical values, that is the much reduced standard error and the tighter confidence intervals obtained with the non-linear logistic regression method, occurs because the logistic model attributes appropriate weights to various biomarkers associated with cardiovascular disease. Indeed, it is generally accepted that the relationship between risk factors or biomarkers is unlikely to be simply additive and that the effect of the association of two or more risk factors (Toumpoulis, et al., 2005), or biomarkers, can be much more or much less than simply summing the individual biomarkers' contributions. This factor might especially be the case when each biomarker has an important impact on predicted status, or if the biomarkers on the panel can be grouped into various classes that constitute sub-categories of the disease.

Application of the LOC assay system, which may accommodate detection of promising biomarkers in bodily fluids in a multiplexed fashion, in conjunction with a cardiobioindex-driven method for biomarker validation, is shown in FIG. 20A-FIG. 20C whereby a total of 9 important protein biomarkers are measured simultaneously appears to be a promising strategy for identification of biomarker diagnostic utility. The development of such multiplexed LOC methods allows for the automated measurement of numerous relevant biomarkers using a single sample and a common miniaturized measurement platform. Collectively, these attributes are combined here to facilitate the future practical measurement of such proteins as a point-of-care diagnostic tool.

### Example 3

### Saliva-Based Classification of Coronary Artery Disease (CAD)

### I. Materials and Methods

Patient Recruitment and Sample Collection—The rights of all human subjects involved in these studies were protected by each of the institutional review boards of the three participating research sites. In all cases, informed consent was granted prior to sample collection. To ensure privacy rights of all study participants, all samples were tested de-identified UL cohort—collection of unstimulated saliva. Thirty-five study participants were recruited at the University of Louisville (UL), Louisville, Ky. From those study participants, 13 had verifiable ASHD and 22 had no CVD. Each subject provided ˜2 mL of unstimulated saliva in sterile, plastic specimen tubes. Subjects rinsed their mouth with water before sample collection, so as to remove any foreign matter that may be present. Samples were positioned upright in a styrofoam test tube holder in a cooler that contained dry ice and then transferred to storage at −70° C. until shipment to The University of Texas at Austin (UT) for analysis with proteomic μ-array chip and LOC system. The University of Kentucky (UK-Lexington, Ky.) cohort consisted of 13 subjects, 4 healthy (with no CVD) and 9 ASHD patients, diagnosed with acute myocardial infarction (AMI). Each subject donated ˜2 mLs of paraffin-stimulated whole saliva into a sterile plastic specimen tube. All samples were aliquoted and stored at −70° C. until testing locally for CRP using the Luminex® —based approach. Duplicate aliquots of the same samples were shipped frozen on dry ice to UT for analysis of CRP content with the LOC system.

LOC-based measurement of CRP and multiplexed LOC tests—Previous studies have described the design, fabrication, and testing of nano-bio-chip LOC structures whereby immunoassays are performed on chemically-sensitized beads within biochip structures (flow cells) with integrated fluid handling and optical detection capabilities (Christodoulides et al., 2002; Christodoulides et al., 2005b) The total time for the LOC-based assay for salivary CRP is 10 minutes. Beads coated with a CRP-specific capture antibody (Accurate Chemical, Westbury, N.Y.) are sequentially exposed to the CRP antigen (as a protein standard (Cortex Biochemicals, San Leandro, Calif.) or in the saliva sample) and then to a detection antibody (Accurate Chemical, Westbury, N.Y.) conjugated to Alexafluor-488 to produce a [CRP]-dependent fluorescent signal within and around the bead. The biochip hosting the bead-based assay allows for the microscopic evaluation of fluorescent signals generated within the array after each assay run. The final image of the bead array is captured by a charge-coupled device (CCD) video chip and digitally processed and analyzed with Image Pro Plus software. The data is analyzed using a four parameter logistic equation process within the SigmaPlot® environment to generate a dose-response curve derived from the CRP standards, which is then used to interpolate the CRP concentrations in the samples.

In this study, LOC-based immunoassays were used for the multiplexed detection of the following eight biomarkers: CRP, IL-6, monocyte chemoattractant protein-1 (MCP-1), IL-1β, myeloperoxidase (MPO), sCD40L, TNF-α and human serum albumin (HSA). Reagents used for CRP assay component of the multiplexed test were such as those described in the single biomarker test described above. Analyte specific capture antibodies for the remaining analytes included: monoclonal antibodies (mAbs) MAB206 and MAB201 (R&D Systems, Minneapolis, Minn.) for IL-6 and IL-1β, respectively; mAbs K86005M and H45700M for MPO and HSA (BIODESIGN International, Saco, Me.), respectively; mAb CMI030 (Cell Sciences, Canton, Mass.) for TNF-α; mAb MCA2486 (AbD Serotec, Kidlington, Oxford, UK) for MCP-1 and mAb 30B4 (HyTest Ltd, Turku, Finland) for sCD40L. The 40 minute LOC multiplexed assay includes a 20-minute incubation with the analyte and a 10-minute incubation with a cocktail of fluorescent detection antibodies, each specific for each of the analytes targeted, followed by a 5 minute wash with PBS. CRP, IL-6, MCP-1, IL-1β, MPO, sCD40L, TNF-α and HSA antigens were purchased from Accurate Chemical, Westbury, N.Y., eBioscience, San Diego, Calif., AbD Serotec, Kidlington, Oxford, UK, Cell Sciences, Canton, Mass., BIODESIGN International, Saco, Me., Cell Sciences, Canton, Mass., BD Biosciences, San Jose, Calif. and Sigma-Aldrich, St. Louis, Mo., respectively. Detection antibodies for CRP, IL-6, MCP-1, IL-1β, MPO, sCD40L, TNF-α and HSA analytes were BMDA29 (Accurate Chemical and Scientific Corp, Westbury, N.Y.), CMI302 (Cell Sciences, Canton, Mass.), GTX18677 (Genetex, San Antonio, Tex.), AB 201—NA (R&D Systems, Minneapolis, Minn.), K50891R (BIODESIGN International, Saco, Me.), 2A3 (HyTest Ltd, Turku, Finland), CMI031 (Cell Sciences, Canton, Mass.) and H86611M (BIODESIGN International, Saco, Me.), respectively.

μ-array measurements-Allied Biotech's (Ijamsville, Md.) antibody-based human cardiovascular α-array kit was used in this study to test for the presence of 14 different cardiovascular markers TNFα, interferon (INF)-γ, sCD-40L, BNP, FABP, cardiac troponin I (cTnI), CKMB, IL-1β, IL-4, IL-5, IL-6, IL-8, IL-10 and IL-13 in unstimulated salivas collected at UK. Each slide in the kit contained 16 identical arrays of 14 capture antibodies in quadruplicate and supported the analysis of up to sixteen 40-μL samples. A cocktail of biotinylated detection antibodies combined with the capturing antibodies spotted on the slide, comprised the antibody pairs to detect the biomarkers. Biomarker detection was achieved with the addition of Streptavidin-Cy5 conjugate, for a fluorescent-based detection. Positive and negative controls spotted within each array allowed for assay validation. A μ-array scanner (GenePix Personal 4100A, Molecular Devices Corporation, Sunnyvale, Calif.) was used, in conjunction with compatible image analysis software (GenePix Pro 6.0, Molecular Devices Corporation, Sunnyvale, Calif.), to determine the background-subtracted signal of each spot. The quadruplicates were then averaged to quantify the specific signal to noise ratio for each biomarker on the array. Using the average signal intensity of a negative control sample (3% BSA/PBS) as a baseline allowed for the determination of the relative abundance of each biomarker in each of the samples. In this study, μ-array assays for IFN-γ, FABP, cTnI and CKMB produced no signal in response to either protein standard or sample and were thus assumed as non-functional.

Luminex® measurements—Beadlyte technology using a Luminex® IS-100 instrument (Luminex Corp. Austin, Tex.) was employed for the measurement of CRP in stimulated saliva. The reagent kit for the CRP assay was acquired from Linco Research (St. Charles, Mo.) and procedures were followed according to the manufacturer's instructions.

Procedures and Statistics for the Determination of Cardiobioindex, Sensitivity, and Specificity of Single Biomarkers and Biomarker Combinations

The following steps were completed to establish the utility of the biomarkers in saliva for the classification of CAD patients:

First, the biomarkers levels for all healthy controls and CAD case samples were measured and results were recorded. If the assay was semi-quantitative, as is the case for many μ-array approaches, the relative signal intensities were used to record differences in biomarker levels between samples. If the assay was quantitative, biomarker concentrations interpolated from dose response curves were used to record differences in biomarker levels between samples. For the protein array data, the average of the median spot intensity was calculated for each biomarker, and served as an independent variable in the analysis. For the LOC and Luminex data, the concentration of the biomarkers was extracted based on a 4-parameter logistic curve using SigmaPlot®.

Second, the relative levels of each biomarker were normalized for all subjects and a dichotomous approach was used to divide the sample population into two well-phenotyped, “control” and “CAD”, populations.

Third, both linear and logistic regression models were used for the analysis of data. In the case of linear regression, all weights of the biomarkers were assumed to be the same allowing for the simple addition of the biomarker contributions. In contrast, the logistic regression model intrinsically attributed different weights for each of the biomarkers. Statistica 5.5 software platform was used for the logistic regression, with the maximum likelihood as the loss function. The method chosen for the estimation was a Hooke-Jeeves pattern moves, with a maximum number of iterations set at 50 and a convergence criterion of 0.0001. Values of the area under the curve (AUC), or C statistic were computed, as well as the standard error (SE), and applied using a two tailed p-value at the 95% confidence level. The biomarker utility index, or cardiobioindex, for each biomarker or combination (panel) of biomarkers was defined simply by the AUC or the C-statistic.

Fourth, the sensitivity and specificity for single biomarker and biomarker aggregates were measured. Here, the best ROC curve from a variety of inputs (biomarkers) is used along with definition of the beta weights to create an index that can be used to classify the patients. The predicted values are used to construct ROC curves of the total positive response (TPR) as a function of false positive rate (FPR), using analyse-it (Analyse It Software, Ltd).

### II. Results

Eleven out of the 15 cardiac biomarkers tested with the protein μ-array and LOC methodologies, were detectable in the salivas collected from healthy controls and CAD patients from the UL cohort. The relative concentration range and median concentrations of biomarkers TNFα, sCD-40L, BNP, IL-1β, IL-4, IL-5, IL-6, IL-8, IL-10, IL-13 and CRP, between control and diseased patient groups demonstrated a significant overlap when data were plotted as a Box and Whisker chart (FIG. 21).

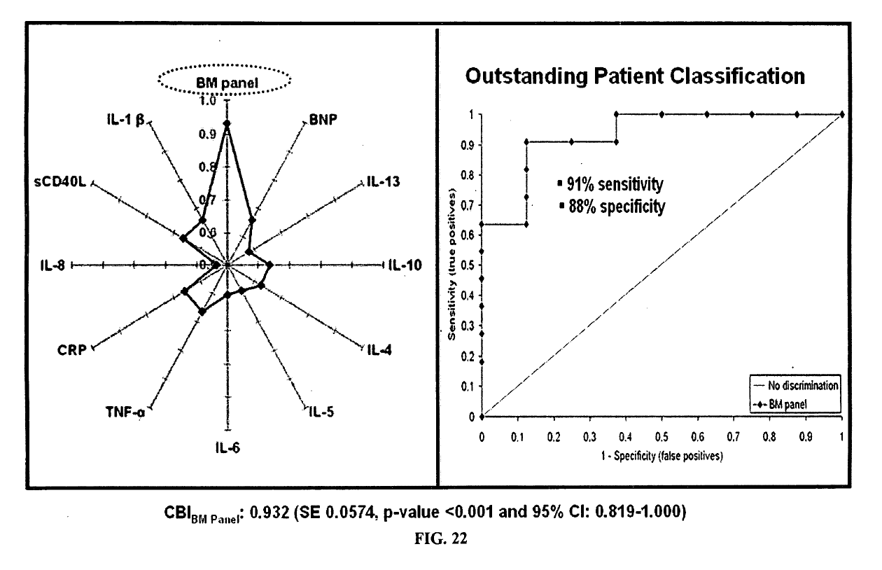

To promote a better evaluation of the biomarker capability to discriminate between control and diseased populations, ROC curve and logistic regression analysis of the data were applied. Single biomarkers TNF-α, sCD40L, BNP, IL-1β, IL-4, IL-5, IL-6, IL-8, IL-10, IL-13 (as measured by proteomic μ-array chip) and CRP (as measured by LOC) produced cardiobioindices in the range of 0.534-0.665 (FIG. 22A).

The inventors next considered the global, or aggregate, biomarker expression profiles and compared them with the classification indices for single biomarkers. Here the biomarker panel consisting of biomarkers TNF-α, sCD40L, BNP, IL-1β, IL-4, IL-5, IL-6, IL-8, IL-10, IL-13 and CRP provides a significantly superior cardiobioindex of 0.932 (SE 0.0574, p-value <0.001 and 95% CI: 0.819-1.000) (FIG. 22A). Furthermore, the combination of the 11 biomarkers contributes to the classification of control and ASHD patients with 91% sensitivity and 88% specificity (FIG. 22B). These values, as derived from multiplexed saliva analysis, are considered to be excellent for classifying patients with CVD.

In addition to the logistic regression, the inventors also explored the C statistic values obtained from a linear regression model. For the same biomarker panel described above, a cardiobioindex of 0.852 (SE 0.0870, p value <0.0001 and 95% CI: 0.682-1.000), is obtained. The inventors hypothesize that the improved statistical values obtained with the logistic regression method occur because this model attributes appropriate weights to various biomarkers associated with CVD. Indeed, it is generally accepted that the relationship between risk factors is unlikely to be simply additive and that the effect of the association of two or more risk factors (Toumpoulis et al., 2005), or biomarkers in this case, can be much more, or much less, than simply summing the individual biomarkers' contributions. This factor might especially be the case when each biomarker has an important impact on the predicted status, or if the biomarkers on the panel can be grouped into various classes that constitute sub-categories of the disease.

It should be noted that the apparent poor performance demonstrated by some of the emerging biomarkers of CVD tested for in saliva could be a result of inefficiencies associated with the method employed for their measurement. A less sensitive analytical method is not expected to be able to detect, and, thus, measure accurately the less abundant proteins in the complex fluid of saliva. In contrast, an assay with enhanced detection capabilities that can detect the analyte/biomarker in a more sensitive and accurate manner is expected to better detect differences of the biomarker levels between control and disease groups and thus provide a more reliable biomarker validation effort.

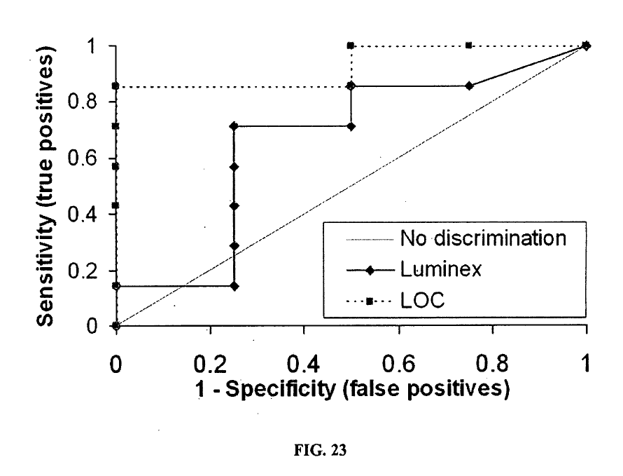

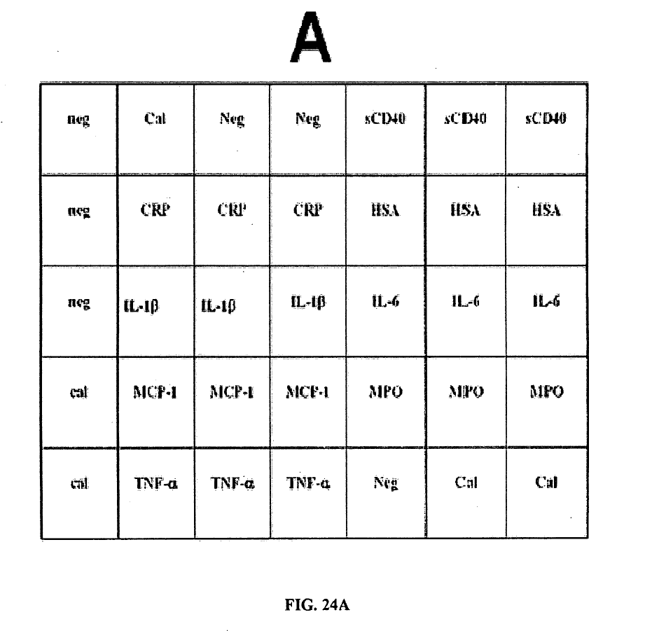

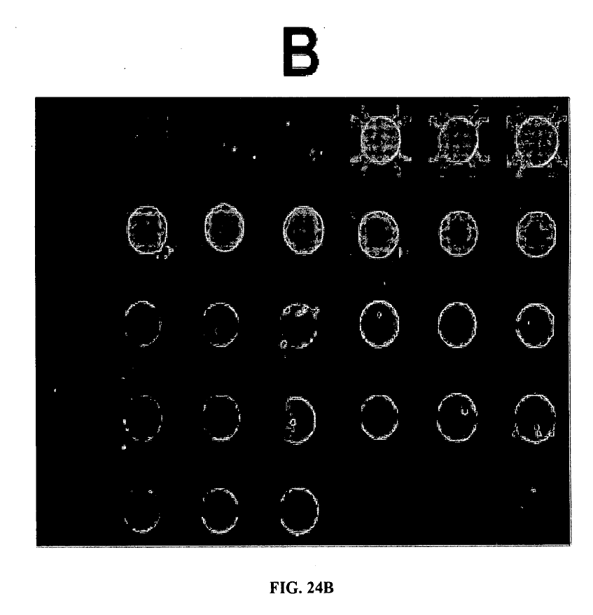

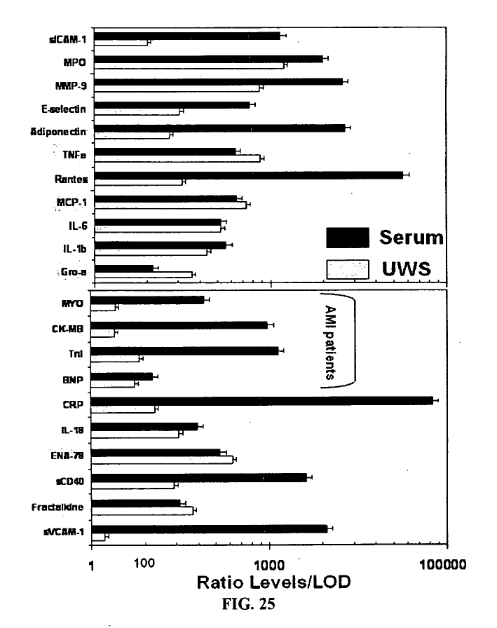

In support of this, when the stimulated saliva samples from the UK cohort are tested for CRP in parallel by LOC and LUMINEX® approaches, a significantly superior classification of ASHD patients is achieved with the biomarker CRP when data achieved with the more sensitive LOC method are considered (FIG. 23). Likewise, when using levels of salivary CRP measured with the Luminex® system (LOD of 80 pg/mL) classification of CAD patients and healthy controls is achieved 71.4% sensitivity and 75% specificity, while, in contrast, LOC (with LD of 10 fg/mL) data provided classification with 85.7% sensitivity and 100% specificity. Here, the Luminex® approach provides a cardiobioindex for CRP at 0.661 (SE 0.1888, p-value 0.1973 and 95% CI: 0.291-1.000), while the counterpart LOC method provides a CRP cardiobioindex of 0.929 (SE 0.0821, p-value <0.0001 and 95% CI: 0.768-1.000).

It is interesting that the CRP cardiobioindex derived from LOC measurements of the salivas from the UK cohort of patients was significantly higher than its counterpart from the UL cohort (0.929 vs 0.68, respectively). This inconsistency may be attributed either to the fact that different saliva types were tested in each case (stimulated vs unstimulated) or to the fact that, in contrast to the UL cohort in which all ASHD patients were at an earlier stage of the disease, the ASHD patients participating in the UK cohort had all recently suffered an AMI, i.e., characteristic of advanced stage heart disease. The latter hypothesis is consistent with the inventors' recent findings that salivary levels of CRP are significantly elevated in patients with AMI.

In addition to hosting ultra sensitive assays, the miniaturized assay platform of the LOC system, similarly to Luminex® and to the μ-array proteomic chip, accommodates detection of promising cardiac biomarkers in bodily fluids in a multiplexed fashion (FIG. 24). Eight cardiac biomarkers CRP, sCD40L, HSA, IL-1p, IL-6, MCP-1, MPO and TNF-α are detected concurrently by the LOC bead sensors. The development of such multiplexed LOC methods allows for the automated measurement of numerous relevant biomarkers using a single <100 μL saliva sample and a common miniaturized measurement platform. Collectively, these attributes (low detection limits, multi-analyte testing capacity and miniaturized assay platform) promise to facilitate the future practical measurement of such proteins in saliva as a point-of-care diagnostic tool.

### Example 4

### AMI Diagnosis

To initiate defining the protein molecules potentially suitable as biomarkers of cardiovascular disease, the inventors first determined their levels present in oral fluids. Table 2 shows the mean and standard deviations (SD) found for each biomarker evaluated in the oral fluids obtained from the controls. One can see that virtually all analytes were detectable in the three types of fluid samples. However, UWS (unstimulated saliva) provided the highest concentrations of the majority of analytes compared with SWS and OS. In general, concentrations were about two times higher in UWS than SWS and 3-10× higher than OS. The fact that levels of potential cardiovascular biomarkers were detectable in UWS and levels of known cardiac enzymes were low in these fluids was appealing for investigating these levels in more defined cardiovascular disease populations.

TABLE 2
Mean and standard deviations (SD) for each biomarker
evaluated in the oral fluids from the controls

| Analyte | UWS | | SWS | | Os | |
|---|---|---|---|---|---|---|
| (conc./mL) | Mean | SD | Mean | SD | Mean | SD |
| Gro-α (pg) | 230.02 | 439.64 | 151.98 | 319.78 | 72.41 | 325.54 |
| IL-1β (pg) | 42.19 | 76.01 | 20.27 | 36.18 | 0.91 | 1.12 |
| IL-6 (pg) | 87.49 | 138.29 | 38.65 | 50.10 | 0.75 | 1.67 |
| MCP-1 (pg) | 198.94 | 399.34 | 107.96 | 138.78 | 4.24 | 9.54 |
| Rantes (pg) | 9.50 | 19.36 | 4.56 | 6.87 | 0.42 | 0.37 |
| TNFα (pg) | 67.37 | 149.66 | 22.25 | 39.40 | 0.17 | 0.20 |
| Adiponectin | 17.38 | 28.80 | 10.20 | 10.68 | 0.98 | 1.29 |
| (ng) | | | | | | |
| E-selectin (ng) | 2.89 | 10.65 | 2.85 | 9.87 | 2.32 | 6.89 |
| MMP-9 (pg) | 6.65 | 8.53 | 3.31 | 5.60 | 1.60 | 1.35 |
| MPO (ng) | 17.17 | 48.28 | 13.81 | 34.88 | 21.72 | 29.99 |
| sICAM-1 | 80.71 | 64.23 | 47.71 | 40.48 | 12.05 | 11.31 |
| (ng/dL) | | | | | | |
| sVCAM-1 | 16.63 | 11.99 | 17.51 | 13.59 | 6.24 | 5.06 |
| (ng/dL) | | | | | | |
| Fractalkine | 252.40 | 229.90 | 248.10 | 233.03 | 30.86 | 32.04 |
| (pg) | | | | | | |
| sCD-40 (pg) | 24.60 | 50.61 | 23.81 | 45.37 | 7.66 | 14.27 |
| ENA-78 (ng) | 2.41 | 1.91 | 2.03 | 1.83 | 0.02 | 0.04 |
| IL-18(pg) | 149.77 | 83.02 | 124.70 | 103.57 | 96.95 | 49.11 |
| CRP (ng) | 0.59 | 1.95 | 0.34 | 0.86 | 0.54 | 0.84 |
| BNP (pg) | 14.64 | 11.74 | 12.25 | 6.83 | 2.1 | 0.42 |
| TnI (ng) | 0.07 | 0.06 | 0.04 | 0.04 | 0.005 | 0.005 |
| CK-MB (ng) | 0.13 | 0.36 | 0.06 | 0.04 | 0 | 0 |
| MYO (ng) | 0.24 | 0.39 | 0.37 | 0.53 | 0.27 | 0.44 |

The inventors then compared the relative levels of 21 proteins as measured in the serum and saliva samples collected from the study participants with respect to the performance of the corresponding assays used for their measurement. As expected, the majority of the analytes were detected at higher ratios in serum. Here, the majority of the analytes were at least 100× above the limit of detection (LOD) of the assay in healthy controls. Advantageously, cardiac enzymes were measured at very low levels in healthy controls, allowing for their distinction from AMI patients.

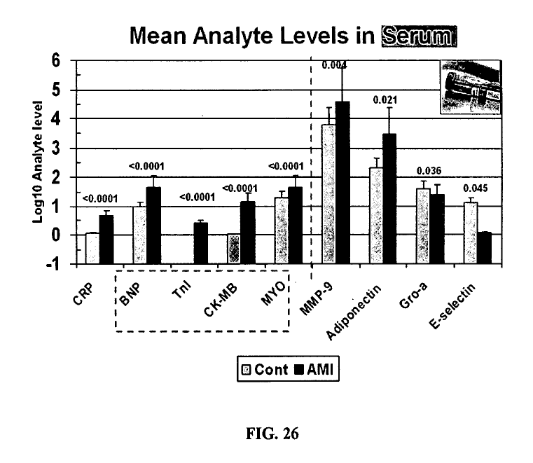

To define a panel of biomarkers that can distinguish AMI from healthy controls, the inventors compared mean analyte levels of all the biomarkers in serum from each group. FIG. 26 shows that 9 biomarkers individually distinguished AMI from health. Not surprisingly, mean concentrations of TnI, CK-MB, MYO and BNP in serum were significantly higher in the AMI than the controls (p<0.0001). Also, serum CRP levels were significantly higher in the AMI than the controls. Of the known serum cardiovascular biomarkers, TnI and CK-MB produced the greatest discriminatory capacity with the mean concentration in the AMI subjects being 1.2-1.5 logs higher than the mean of the controls. The data also revealed four novel biomarkers that distinguish AMI from controls. Mean serum levels of MMP-9 and adiponectin were significantly higher in AMI than controls, whereas Gro-1a and E-selectin were found to be significantly lower in the AMI than the controls.

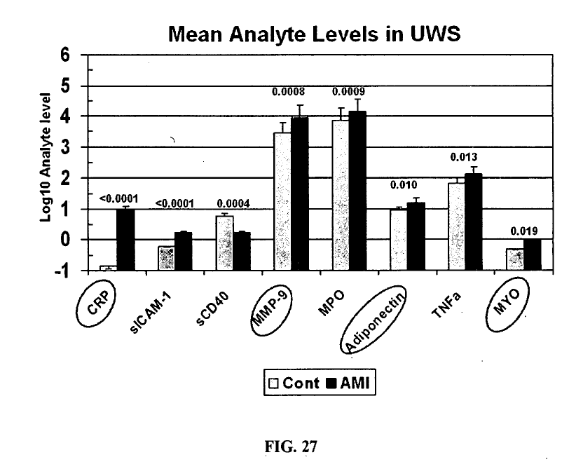

Mean analyte levels in unstimulated saliva (UWS) were likewise determined using Luminex and ELISA. These analyses revealed eight biomarkers with discriminatory capacity between AMI and healthy controls (FIG. 27). As observed with serum, CRP, MMP-9, adiponectin and MYO were significantly higher in the AMI group than the controls. Novel biomarkers of AMI found in UWS were sICAM-1, sCD40, MPO and TNFα. All biomarkers in UWS were significantly higher in the AMI group than the controls, except sCD40L. Overall, these serum and UWS biomarkers, alone and/or together, serve to diagnostically distinguish AMI from healthy controls.

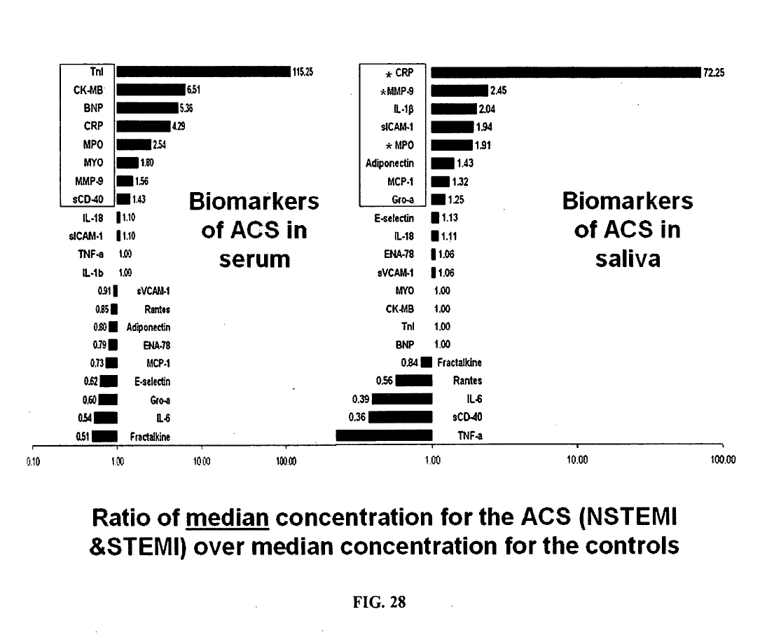

The inventors next compared the ratios of the median concentrations of biomarkers in AMI and healthy controls, in serum and saliva, as a means to identify those biomarkers that are up- or down-regulated with AMI, in each bodily fluid. FIG. 28 shows that serum biomarkers cTnI, CK-MB, BNP, CRP, Myoglobin, MMP9 and sCD40L exhibited significantly higher median concentrations in the serum of AMI patients than in healthy controls. In saliva, biomarker CRP showed the highest ratio in median concentration of AMI/healthy control, followed by MMP9, IL-1b, sCD40L, MPO, adiponectin, MCP-1 and Gro-A. A direct comparison of the serum and saliva biomarkers showed that the two fluids shared biomarkers CRP, MMP9 and MPO as the top ranked biomarkers. From these three biomarkers, CRP and MPO have been approved by FDA for clinical use.

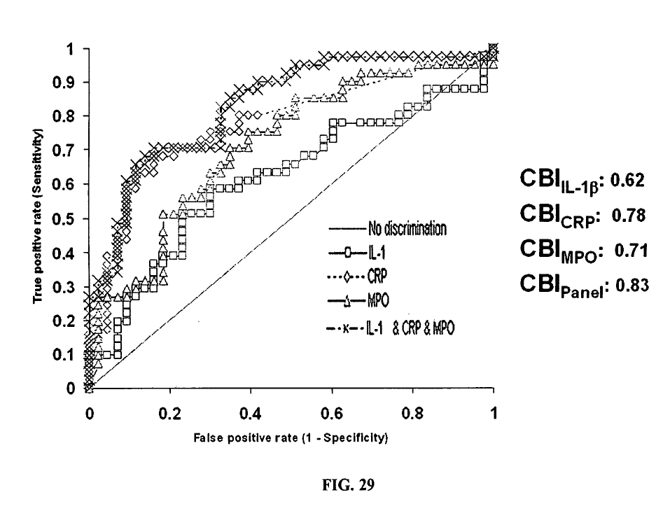

In order to promote a better evaluation of the biomarker capability to discriminate between control and diseased populations, logistic regression and ROC analysis of the data were then applied. Once again, the term cardiobioindex was applied as a means to describe the ability of each biomarker (or combination of biomarkers) to discriminate between healthy controls and cardiac (AMI) patients. Representative data for some of the top ranking biomarkers in saliva are shown in FIG. 29. Here, biomarkers IL-1β, CRP and MPO demonstrated CBIs of 0.62, 0.78 and 0.71, respectively. Their combination as a panel produced a CBI of 0.83, suggesting that a multi-analyte screening approach provides improved diagnostic capabilities.

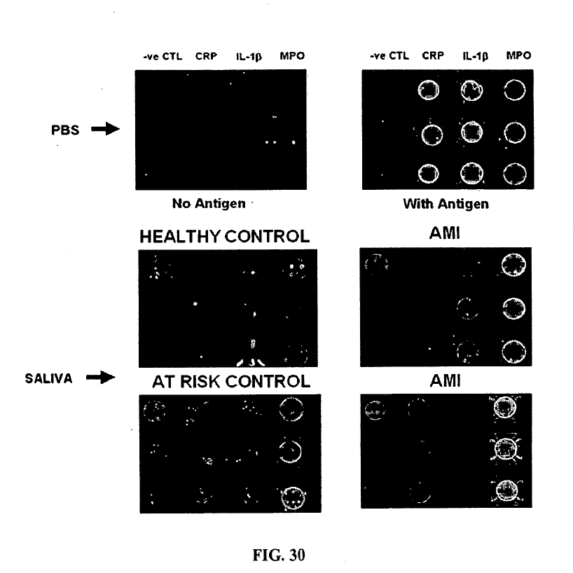

With that in mind, the inventors took advantage of the multi-analyte testing capacity of their LOC sensor and developed the relevant multiplexed test for the 3 salivary biomarkers CRP, IL-1β and MPO. FIG. 30 shows the results achieved on the LOC sensor, first in PBS and then in saliva of healthy controls, at risk controls and AMI patients. Consistent, with the inventors' previous findings, all 3 biomarkers demonstrated significant elevations in the AMI patients, as revealed by the increase in signal intensities derived on the relevant, analyte-specific bead sensors.

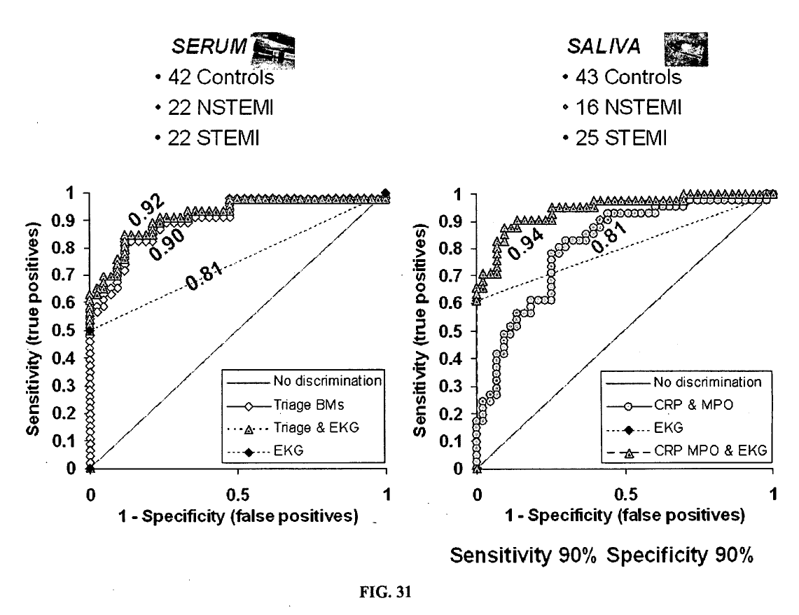

To create a more sensitive and specific diagnostic test for AMI than that offered by the aggregate top-ranked salivary biomarkers CRP, MPO and IL-1b (CBI of 0.83), the inventors considered the established, and now in place, criteria for diagnosis of AMI, such as EKG and serum-based cardiac enzymes. Upon investigation of different combinations of biomarkers and tests, ascertained through logistic regression and ROC analysis of the data, the inventors had identified that the combination between the two top ranking salivary biomarkers (CRP and MPO) in conjunction with EKG exhibited outstanding AMI diagnostic capabilities (FIG. 31). Here, results achieved in the sera of 84 study subjects (42 healthy controls, 46 AMI-23 NSTEMI, 23 STEMI) are reported and compared to the optimal saliva-based tests. In serum, as expected, EKG had a CBI of 0.75, as it failed to identify the NSTEMI component of the AMI group. The TRIAGE biomarkers (cTnI, myoglobin and CK-MB) considered in aggregate, were associated with a CBI of 0.90, and then their combination with EKG, derived a CBI of 0.92. Again, when salivary biomarkers CRP and MPO were considered together, a CBI of 0.81 was achieved. However, the combined use of CRP and MPO in saliva, in conjunction with EKG, produced an excellent CBI of 0.94. Here, this panel demonstrates discrimination between healthy and cardiac disease with 90% sensitivity and 90% specificity. These exciting findings demonstrate that salivary biomarkers, when used in conjunction with EKG, indeed offer significant utility for the diagnosis of AMI at a level comparable to the established and widely accepted serum-based tests.

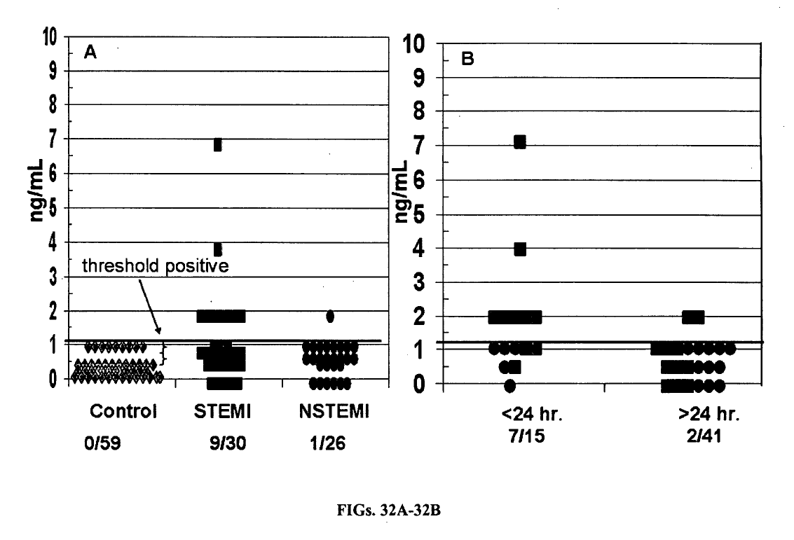

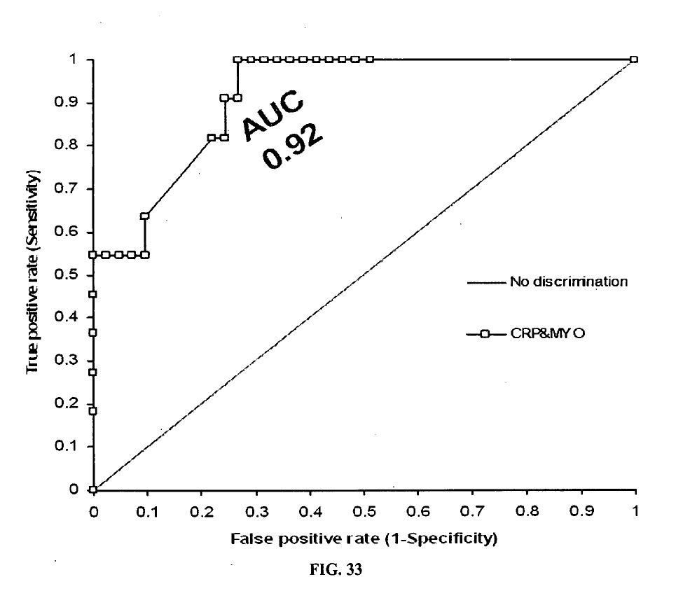

A certain embodiment of the present invention is the evaluation of the time course of elevation of certain biomarkers of AMI. As discussed earlier, the temporal pattern of marker protein release is of diagnostic importance. The inventors thus focused on the exemplary biomarker myoglobin which, in serum, is known to be released within 24 hours of onset of symptoms of AMI, time after which reported levels return to baseline. To more carefully examine the diagnostic utility of myoglobin, the inventors set a threshold value of 2 standard deviations above the mean level of the control group (i.e., 1.2 ng/ml), consistent with the practice of clinical pathology laboratories in defining abnormal values in the population. With this threshold set, the inventors identified 18% of the AMI subjects (10/56) and 30% of the STEMI subjects (FIG. 32A). None of the controls had UWS myoglobin levels above the threshold yielding a specificity of 100%. Since myoglobin levels peak in serum between 4-8 hrs after onset of ACS, the inventors further delineated the UWS myoglobin profile for subjects who enrolled in the inventors' study within 24 hr of onset of symptoms. Panel B shows that 47% ( 7/15) of persons presenting with ACS within 24 hrs had UWS myoglobin above the threshold for diagnosis, again with 100% specificity. The inventors extended the diagnostic utility of myoglobin by pairing it with CRP (the UWS analyte with the highest diagnostic discriminatory capacity) and performed logistic regression and ROC analysis. FIG. 32B shows that these two biomarkers produce a CBI of 0.92 which is equivalent to the CBI produced by the combined use of serum TnI, serum CK-MB and EKG. Thus, these data indicate that UWS biomarkers are diagnostically equivalent with the current measures used for the diagnosis of ACS in hospitals throughout the U.S.

### REFERENCES

The following references, to the extent that they provide exemplary procedural or other details supplementary to those set forth herein, are specifically incorporated herein by reference.
U.S. Patent Appln. 2002 0138208 A1
U.S. Patent Appln. 2002 0193950
U.S. Patent Appln. 2003 0004402
U.S. Patent Appln. 2003 0055615 A1
Aguirre et al., Crit. Rev. Oral Biol. Med., 4(3-4):343-350, 1993.
Ali et al., Analytical Chem., 75(18):4732-4739, 2003.
American Heart Association, Heart and Disease Statistics—2004.
Anderson and Anderson, Molecul. Cell. Proteomics, 1(11):845-867, 2002.
Anderson, J. Physiology-London, 563:23-60, 2005b.
Anderson, Molecul. Cell. Proteomics, 4(10):1441-1444, 2005a.
Aps and Martens, Forensic Sci. Int., 150(2-3):119-31, 2005.
Arab et al., J. Amer. Coll. Cardiol., 48(9):1733-1741, 2006.
Baumann et al., J. Biol. Chem., 265(36):22275-22281, 1990.
Baumann et al., Molec. Biol. Med., 7(2):147-159, 1990.
Bernard et al., Lab. Chip, 5:261-269, 2005b.
Blake et al., Europ. Heart J, 24:116-116, 2003.
Chia et al., Oral Microbiol. Immunol., 15(2):131-8, 2000.
Christodoulides et al., Analytical Chem., 74(13):3030-3036, 2002.
Christodoulides et al., Clinical Chem., 51(12):2391-2395, 2005a.
Christodoulides et al., Lab. Chip, 5(3):261-9, 2005b.
Cone, Ann. NY Acad. Sci., 694:91-127, 1993.
Curey et al., Analy. Biochem., 293(2):178-184, 2001.
Curtis et al., Trends in Biotechnology, 23(8):429-435, 2005.
de Lemos et al., Lancet., 362(9380):316-322, 2003.
de Lemos et al., NE J. Med., 345(14):1014-1021, 2001.
Depraetere et al., Agents and Actions, 34(3-4):369-375, 1991.
DeStefano et al., BMJ, 306(6879):688-91, 1993.
Desvarieux et al., Circulation, 111(5):576-82, 2005.
Do et al., Molecules and Cells, 22(3): 254-261, 2006.
Donahue et al., Amer. Heart J, 152(3):478-485, 2006.
Ferguson, J Dental Res., 66(2):420-424, 1987.
Floriano et al., Biosens. Bioelect., 20(10):2079-2088, 2005.
Fox et al., Adv. Exp. Med. Biol., 438:909-15, 1998.
Fu and Van Eyk, Expert Rev. Proteom., 3(2):237-249, 2006.
Fung et al., Expert Rev. Proteom., 2(6):847-862, 2005.
Ganaphthi et al., J. Immunol., 147(4):1261-1265, 1991.
Ganter et al., Embo. J, 8(12):3773-3779, 1989.
Goodey et al., J. Amer. Chem. Soc., 123(11):2559-2570, 2001.
Goodey et al., J. Amer. Chem. Soc., 125(10):2870-2871, 2003.
Grisius et al., J. Rheumatol., 24(6):1089-1091, 1997.
Haeckel and Hanecke, Ann. Biol. Clin. (Paris), 51(10-11):903-10, 1993.
Haeckel, J Clinical Chem. Clinical Biochem., 27 (4):223-226, 1989.
Hanemaaijer et al., Matrix Biol., 17(8-9):657-65, 1998.
Haraszthy et al., J. Periodontol., 71(10):1554-60, 2000.
Harris et al., Amer. J. Med., 106(5):506-512, 1999.
Herrmann, Cardiovas. Res., 60(2):220-222, 2003.
Hodinka et al., Clin. Diag Lab. Immun., 5(4):419-426, 1998.
Hortin et al., Clinical Chem., 52:1218-1222, 2006.
Horwich et al., Circulation, 108(7):833-838, 2003.
Huang, J. Immunol. Methods, 255(1-2):1-13, 2001.
Jain, “Statistical Pattern Recognition: A Review”, IEEE Transactions on Pattern Analysis and Machine Intelligence, Vol. 22, No. 1, January 2000
Jarvis et al., BMJ, 321(7257):343-345, 2000.
Joshipura et al., J. Dent. Res., 75(9):1631-6, 1996.
Jung et al., Biometrical J, 48(2):245-254, 2006.
Kaufman and Lamster, J. Clin. Periodontol., 27(7):453-65, 2000.
Kragelund et al., NE J. Med., 352(7):666-675, 2005.
Kushner and Rzewnicki, Baillieres Clinical Rheumat., 8(3):513-530, 1994.
Lam et al., J Cardiology, 108(1):12-19, 2006.
Lamster et al., J. Periodontol., 74(3):353-9, 2003.
Lavigne et al., J. Amer. Chem. Soc., 120(25):6429-6430, 1998.
Lee et al., Crit. Rev. Clinical Lab. Sci., 44(1):87-114, 2007.
Li et al., Biosens. Bioelect., 21:574-580, 2005a.
Li et al., Clin. Cancer Res., 10(24):8442-50, 2004a.
Li et al., J. Dent. Res., 83(3):199-203, 2004b.
Li et al., J. Microelectromech., Sys., 14(6), 2005b.
Libby et al., Circulation, 105(9):1135-1143, 2002.
Lins et al., J. Med. Virol., 77(2):216-20, 2005.
Liszewski, Genetic Engineering News, 26(6): 1-+, 2006.
Liu et al., Analytical Chem., 75(18):4718-4723, 2003.
Loesche, Compendium, 15(8):976, 978-82, 985-6, 1994.
Ludwig and Weinstein, Nature Rev. Cancer, 5(11):845-856, 2005.
Maisel et al., NE J. Med., 347(3):161-167, 2002.
Malamud, Br. J. Med., 305(6847):207-208, 1992.
Mandel, Annals N.Y. Acad. Sciences., 694:1-10, 1993a.
Mandel, Crit. Rev. Oral Biol. Med., 4(3-4):599-604, 1993c.
Mandel, J. Amer. Dental Assoc., 124(1):85-87, 1993b.
Mandel, J Oral Path. Med., 19(3):119-125, 1990.
Manz et al., Sensors Actuators B-Chemical, 1(1-6):244-248, 1990.
Mattila et al., BMJ, 298(6676):779-81, 1989.
Mayr et al., Molecul. Cell. Proteomics, 5(10):1853-1864, 2006.
McCleskey et al., Angewandte Chemie-International Ed., 42(18):2070-2072, 2003b.
McCleskey et al., J. Amer. Chem. Soc., 125(5):1114-1115, 2003a.
Michel et al., J Cardio-Thoracic Surg., 23:684-687, 2003.
Mogi et al., Arch. Oral Biol., 38(12):1135-9, 1993.
Morrow et al., Jama-J. Amer. Med. Assoc., 286(19):2405-2412, 2001.
Nakamura et al., Rev. Cardiovasc. Medicine, Suppl. S22-S33, 2004.
Napoli et al., Heart, 89(6):597-604, 2003.
Noack et al., J. Periodontol., 72(9):1221-1227, 2001.
Nogueira et al., Infect. Immun., 73(9):5675-84, 2005.
Office of Technology Commercialization 27(21) IPQ for the The University of Texas at Austin Biological Sciences
Office of Technology Commercialization Page 20 of 27 IPQ for the The University of Texas at Austin Biological Sciences
Omenn, Proteomics, 6(20):5662-5673, 2006.
PCT Appln. WO 2000/004372
PCT Appln. WO 2001/031580
PCT Appln. WO 2001/006239
PCT Appln. WO 2001/006244
PCT Appln. WO 2001/006253
PCT Appln. WO 2001/055701
PCT Appln. WO 2001/055702
PCT Appln. WO 2001/055703
PCT Appln. WO 2001/055704
PCT Appln. WO 2001/055952
PCT Appln. WO 2002/061392
PCT Appln. WO 2003/090605
PCT Appln. WO 2004/009840
PCT Appln. WO 2004/072097
PCT Appln. WO 2004/072613
PCT Appln. WO 2005/059551
PCT Appln. WO 2005/083423
PCT Appln. WO 2005/085796
PCT Appln. WO 2005/085854
PCT Appln. WO 2005/085855
PCT Appln. WO 2005/090983
PCT Appln. WO 2007/002480
PCT Appln. WO 2007/005666
PCT Appln. WO 2007/134189
PCT Appln. WO 2007/134191
Quackenbush, Nature Genetics, 32:496-501, 2002.
Rhodus et al., Cancer Detect. Prev., 29(1):42-5, 2005.
Ridker, Amer. Heart J., 148(1): S19-S26, 2004.
Ridker, NE J. Med., 352(1):20-28, 2005.
Ridker et al., NE J. Med., 347:1557-1565, 2002.
Rifai and Ridker, Clin. Chem., 49:666-669, 2003.
Rodriguez et al., Plos. Medicine, 2(7):663-672, 2005.
Ross, NE J Med., 340(2):115-126, 1999.
Sabatine et al., Circulation, 105(15):1760-1763, 2002.
Schramm et al., J Analytical Toxicol., 16(1):1-9, 1992.
Situma et al., Analytical Biochem., 340(1): 123-135, 2005.
Slavkin, J. Amer. Dental Assoc., 129(8): 1138-1143, 1998.
Sluss, Point of Care, 5:38-46, 2006.
St John et al., Arch. Otolaryngol. Head Neck Surg., 130(8):929-35, 2004.
Stephan et al., Prostate, 66(6):651-659, 2006.
Stroehle et al., J. Parasitol., 91(3):5.61-3, 2005.
Suzuki et al., J. Clin. Microbiol., 43(9):4413-7, 2005.
Svojanovsky et al., J. Pharm. Biomed. Anal., 20(3):549-555, 1999.
Toennes et al., Forensic Sci. Int., 152(2-3):149-55, 2005.
Toniatti et al., Molec. Biol. Med., 7(3):199-212, 1990.
Toumpoulis et al., Europ. J Cardio-Thoracic Surg., 27:128-133, 2005
Tudos et al., Lab. Chip, 1(2):83-95, 2001.
Vasan, Circulation, 113(19):2335-2362, 2006.
Venugopal et al., Amer. J. Pathol., 166(4):1265-1271, 2005.
Verhoeckx et al., Proteomics, 4(4):1014-1028, 2004.
Verpoorte and De Rooij, Proceed. Ieee, 91(6):930-953, 2003.
Walsh et al., J. Anal. Toxicol., 27(7):429-39, 2003.
Whitesides, Small, 1(2):172-179, 2005.
Wu et al., Arch. Intern. Med., 160(18):2749-2755, 2000.
Wu, Point of Care, 5:20-24, 2006.
Yang et al., Lab. Chip., 5(10):1017-23, 2005.
Zevin et al., Drug Alcohol Depend., 60(1):13-8, 2000.
Zhu et al., Genome Biol., 7(11), 2006.
Zolg, Molecul. Cell. Proteomics, 5(10):1720-1726, 2006.

What is claimed:

1. A method for assessing cardiovascular disease status in a subject comprising the steps of:
(a) measuring a biomarker level in a sample from a subject, wherein the biomarker is two or more of CRP, IL1β, IL-13, cTnI, BNP, FABP, CK-MB, IL-6, IL-8, IL-10, TNF-α, CD40L, IFN-γ, myoglobin, MMP9, sICAM-1, myeloperoxidase, IL-4, and/or IL-5;
(b) evaluating biomarker levels with respect to a scoring index, wherein evaluation comprises:
(i) assigning an index to each biomarker or combination of biomarkers based on its/their measured capacity to discriminate between cardiac healthy subjects and cardiac disease patients,
(ii) establishing a threshold level of the biomarker with an index greater than 0.8 to discriminate cardiac healthy subjects from cardiac disease patients; and
(c) determining a value representative of the cardiovascular disease status of the subject based on the evaluation of subject's biomarker.

2. The method of claim 1, wherein the sample is a saliva sample.

3. The method of claim 2, wherein the saliva sample is a stimulated saliva sample.

4. The method of claim 1, wherein the threshold level for a biomarker indicates the presence or absence of a biomarker.

5. The method of claim 1, wherein the threshold level indicates a risk level division in which the measured biomarker level falls.

6. The method of claim 1, wherein the threshold level is determined by the steps of:
(a) obtaining a sample from each of a plurality of subjects including cardiac healthy subjects and cardiac disease subjects at risk of or having cardiovascular disease;
(b) quantifying the level of the biomarkers in each sample;
(c) comparing the level between the cardiac healthy subjects and the cardiac disease subjects;
(d) identifying and selecting a biomarker that distinguish the cardiac healthy subjects from the cardiac disease subjects; and
(e) determining a threshold level for the selected biomarker based on discriminatory concentration for the selected biomarker.

7. The method of claim 1, wherein the assessing cardiovascular status is classification of risk for cardiovascular disease, diagnosis of acute myocardial infarction (AMI), assessment of risk for a second AMI, and/or patient prognosis after AMI.

8. The method of claim 1, wherein assessing cardiovascular disease status is diagnosis of AMI, whereas in step (a) the sample is serum and the biomarker is two or more of cTnI, CK-MB, BNP, myoglobin, and/or CRP.

9. The method of claim 1, wherein assessing cardiovascular disease status is diagnosis of AMI, whereas in step (a) the sample is saliva and the biomarker is two or more of CRP, IL-1β, myeloperoxidase, myoglobin, MMP9, and/or sICAM-1.

10. A method of establishing a cardiobioindex comprising the steps of:
(a) obtaining a plurality of samples from a first and second population of subjects, wherein the first population has a normal cardiac status and the second population has a cardiovascular condition;
(b) quantifying the level of a factor in each sample;
(c) comparing the factor levels between the healthy subjects and the cardiac patients;
(d) determining the cardiobioindex of the factor by logistic regression and ROC analyses; and
(e) utilizing factors with cardiobioindex greater than 0.8 for cardiac diagnostics.

11. The method of claim 10, wherein the factor is a biomarker, BMI, blood pressure, total cholesterol, lipid ratio, or combinations thereof.

12. The method of claim 11, wherein the biomarker is LDL, HDL, C-reactive protein (CRP), adiponectin, Apolipoprotein A (ApoA), Apolipoprotein B (Apo B), E-selectin, IL-1α, IL-1β, IL-4, IL-5, IL-6, IL-1β, IL-10, IL-13, IL-18, creatinine kinase-MB (CK-MB), B-natriuretic peptide (BNP), FABP (cardiac fatty acid protein), TNF-α, MCP-1, MMP-9, MPO, Intercellular Adhesion Molecule (ICAM), Vascular Cellular Adhesion Molecule (VCAM), sCD40L, ENA78, fractalkline, PIGF, PAPP-A, RANTES, sCD40L, vWF, D-dimer, IMA, FFAu, Choline, cTnT, Cardiac troponin I (cTnI), Myoglobin, NT-proBNP, MMP or a combination thereof.

13. The method of claim 10, wherein the cardiovascular disease is atherosclerotic heart disease (ASHD), acute coronary syndrome, cardiomyopathy, microvascular angina, hypertension, ST elevated myocardial infarction, non-ST elevated myocardial infarction, acute myocardial infarction (AMI), coronary heart disease (CHD) or coronary artery disease (CAD).

14. The method of claim 10, wherein the sample is a body fluid.

15. The method of claim 14, wherein the body fluid is serum, saliva, urine, blood, blood plasma, or cerebrospinal fluid.

16. The method of claim 10, wherein the level is quantified by a detection device.

17. The method of claim 16, wherein the detection device is lab-on-a-chip.
